(19) United States
(12) Patent Application Publication
(10) Pub. No.: US 2002/0120200 A1
(43) Pub. Date: Aug. 29, 2002

(54) Devices, systems and methods for endocardial pressure measurement

(76) Inventors: Brian Brockway, Shoreview, MN (US); Gregory Doten, Crystal, MN (US); Michael Holtz, South St. Paul, MN (US); Michael L. Kalm, Spring Lake Park, MN (US); Scott Kuehn, Bloomington, MN (US); Scott Lambert, East Bethel, MN (US); Gregg Lichtscheidl, Roseville, MN (US); Perry Mills, Arden Hills, MN (US); Oleg Mosesov, Minneapolis, MN (US); Demetre M. Nicoloff, Wayzata, MN (US); Karen Odden, Baldwin, WI (US); Soon Park, Stillwater, MN (US); Christopher Quinn, Minneapolis, MN (US); Alyse Stofer, Woodbury, MN (US); Jeff Taylor, Wyoming, MN (US); Justin VanHee, Minneapolis, MN (US); Lynn Zwiers, Lino Lakes, MN (US)

Correspondence Address: Robert E. Atkinson, CROMPTON, SEAGER & TUFTE, LLC, Suite 895, 331 Second Avenue South, Minneapolis, MN, 55401-2246, US

(21) Appl. No.: 10/077,566

(22) Filed: Feb. 15, 2002

Related U.S. Application Data
(63) Continuation-in-part of application No. 09/159,653, filed on Sep. 24, 1998, now Pat. No. 6,409,674.
(63) Continuation-in-part of application No. 09/825,130, filed on Apr. 3, 2001.
(63) Continuation of application No. 09/264,147, filed on Mar. 5, 1999, now Pat. No. 6,296,615.
(63) Continuation of application No. 09/491,233, filed on Jan. 25, 2000, now Pat. No. 6,379,308.
(63) Continuation of application No. 08/950,315, filed on Oct. 14, 1997, now Pat. No. 6,033,366.
(60) Provisional application No. 60/269,922, filed on Feb. 19, 2001.

(51) Int. Cl. A61B 5/02

(52) U.S. Cl. 600/488; 600/486

(57) ABSTRACT

Endocardial pressure measurement devices, systems and methods for the effective treatment of congestive heart failure and its underlying causes, in addition to other clinical applications.

### FIELD

The subject matter disclosed herein generally relates to medical devices, systems and methods for internal body measurements of respiration, ECG, temperature, heart wall thickness and intra chamber pressure. More specifically, the subject matter disclosed herein relates to medical devices, systems and methods for collecting and using endocardial pressures and related data for diagnosing heart disease. Preferred structures and devices for telemetry and processing are disclosed as well.

### BACKGROUND

Congestive heart failure (CHF) is an end-stage chronic condition resulting from the heart's inability to pump sufficient blood, and is a significant factor in morbidity, mortality and health care expenditure in the United States. The number of CHF patients and the resulting morbidity, mortality, and cost associated with CHF diagnosis and treatment is rapidly increasing. The nearly 6 million CHF patients now treated each year in the U.S. will likely become 10 million by 2007. Based on a 1997 analysis of 29,000 CHF patients, the average cost per-patient per-hospitalization was almost $11,000. Annual expenditures for the inpatient and outpatient care of CHF patients reached $38 billion in 1991, and that annual expenditure rose to an estimated $56 billion in 1999.

There are a variety of underlying conditions that may lead to CHF, and a variety of therapeutic approaches targeting such conditions. The selection of the therapeutic approach, and the parameters of the particular therapeutic approach selected, is a function of the underlying condition and the degree to which it affects the heart's ability to pump blood. Thus, most if not all of the therapeutic approaches to CHF would benefit from a method to measure and monitor, on an ongoing basis, the heart's ability to pump blood. However, a significant limitation in treating patients with CHF, especially those at home, is the inability to assess clinical status readily over time and to make appropriate treatment adjustments, particularly while the patient is at home. CHF patients are thus frequently hospitalized or require extended clinic visits in an attempt to optimize therapy.

A good indicator of the impact of therapy and the heart's ability to pump blood is endocardial blood pressure, such as left ventricle (LV) pressure. Currently, however, there are no approved devices or procedures that are practical for obtaining ongoing, long-term pressure data from the patient at home. For example, invasive procedures, such as heart catheterization procedures, are not practical because long-term monitoring would necessitate repeated invasive procedures. Even noninvasive procedures such as echocardiography are not practical for providing long-term data because patients would not be able to leave the hospital. Consequently, currently available technologies do not permit physicians to assess significant changes in disease states or the effects of treatments on an ongoing basis.

Although there is no practical technology or procedure currently available for obtaining ongoing, long-term pressure data, some patents have proposed systems that attempt to accomplish this. U.S. Pat. No. 5,810,735 to Halperin et al and U.S. Pat. No. 5,904,708 to Goedeke disclose systems for monitoring internal patient parameters such as right ventricular (RV) pressure. However, RV measurements provide only an indirect evaluation of changes in cardiac pumping performance, and thus are not nearly as desirable as data obtained directly from the left ventricle. In addition, although RV end-diastolic pressure corresponds somewhat with LV filling pressure, such measurements can be quite disparate in many cases. Furthermore, RV changes may not be as sensitive in representing treatment effects as direct LV measurements. Consequently, there is a need for a system and method for obtaining accurate, timely and ongoing LV pressure measurements in order to optimize CHF treatment.

### SUMMARY

To address this unmet need, the present invention provides devices, systems and methods for ongoing long-term monitoring of endocardial pressure, such as LV pressure. The embodiments disclosed herein illustrate a variety of ways to measure and monitor endocardial blood pressure, each having associated advantages. By monitoring endocardial blood pressure and other information, the diagnosis and care of a CHF patient may be monitored and modified to better treat CHF and its underlying causes.

Those skilled in the art will recognize that the endocardial pressure measurement devices, systems and methods described herein may have other clinical applications, although not specifically mentioned. In addition, those skilled in the art will recognize that the various embodiments described herein are applicable to human patients as well as animal subjects.

### BRIEF DESCRIPTION OF THE DRAWINGS

FIG. 1A is a side view of an implantable pressure measurement telemetry device, including a remote sensor assembly (RSA) and telemetry unit (TU), in accordance with an exemplary embodiment;

FIG. 1B is a top view of the implantable pressure measurement device illustrated in FIG. 1A;

FIG. 1C is a perspective view of the implantable pressure measurement device illustrated in FIG. 1A, showing the pressure transmission catheter (PTC) catheter of the RSA extending across a heart wall;

FIGS. 1D-1M illustrate longitudinal sectional views of various PTC embodiments;

FIG. 2A illustrates the RSA and TU implanted in a patient, with PTC positioned across the left ventricular heart wall;

FIG. 2B schematically illustrates various possible anatomical implant positions for the RSA;

FIGS. 3A-3C are partially sectioned side views of the RSA and PTC with various delivery configurations for positioning the PTC across the ventricular septum;

FIGS. 4A-4D are side views of the RSA and PTC, schematically illustrating various anchor mechanisms;

FIGS. 5A-5C are sectioned side views schematically illustrating various PTC tip configurations for penetrating the ventricular septum without coring;

FIGS. 6A-6E schematically illustrate a system and method delivering and positioning the PTC across the ventricular septum utilizing an anchor device;

FIGS. 7A-7C schematically illustrate side cross-sectional views of various anchor devices for use with the delivery system shown in FIGS. 6A-6E; and

FIG. 8 is a schematic diagram illustrating an example of a system which communicates with the implantable pressure measurement device, including a home (i.e., local) data collection system (HDCS) and a physician (i.e., remote) data collection system (PDCS);

FIG. 9 is a schematic diagram of the TU and RSA;

FIG. 10 is a schematic diagram of the HDCS;

FIG. 11 is an example of the display of the PDCS;

FIG. 12 is a state diagram illustrating an example of operating states and transitions that may be contained in the TU;

FIG. 13 is a state diagram illustrating an example of operating states and transitions that may be contained in the HDCS; and

FIG. 14 is a state diagram illustrating an example of operating states and transitions that may be contained in the PDCS.

### DETAILED DESCRIPTION

The following description should be read with reference to the drawings wherein like reference numerals indicate like elements throughout the several views. The detailed description and drawings illustrate embodiments by way of example, not limitation.

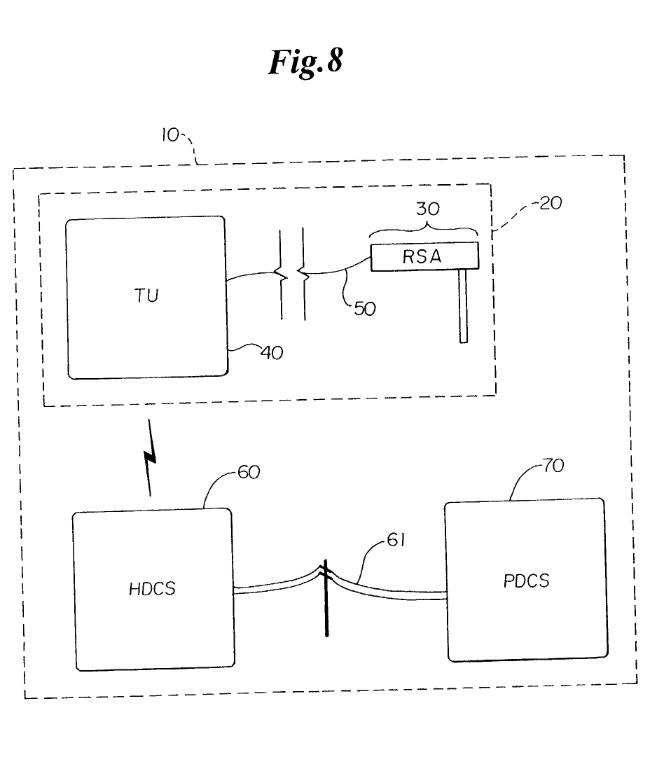

In general, the present invention provides, in an exemplary embodiment, a system 10 for measuring and monitoring endocardial pressure (e.g., LV pressure). The overall system 10 and its function is discussed with reference to FIGS. 8-14.

Brief Description of System

The system 10 includes an implantable telemetry device (ITD) 20, which may be partitioned into a remote sensor assembly (RSA) 30 for measuring endocardial pressure, connected via a lead 50 to a telemetry unit (TU) 40 for telemetering measured pressure data to a receiver located outside the body. An alternative construction mounts all of the ITD 20 in a single housing which may be implanted in any of the positions of the RSA 30 described hereinafter, or directly in a heart chamber. The system 10 also includes a home (i.e., local) data collection system (HDCS) 60 which receives the telemetry signal, optionally corrects for fluctuations in ambient barometric pressure, evaluates the validity of the received signal, and, if the received signal is deemed to be valid, extracts parameters from that signal and stores the data according to a physician-defined protocol.

The system 10 also includes a physician (i.e., remote) data collection system (PDCS) 70 which receives the data signal from the HDCS 60 via a telecommunication system (e.g., the Internet). The PDCS 70 receives the data signal, evaluates the validity of the received signal and, if the received signal is deemed to be valid, displays the data, and stores the data according to a physician-defined protocol. With this information, the system 10 enables the treating physician to monitor endocardial pressure in order to select and/or modify therapies for the patient to better treat diseases such as CHF and its underlying causes.

For example, the system 10 maybe used for assessment of pressure changes (e.g., systolic, diastolic, and LV max dP/dt) in the main cardiac pumping chamber (the LV). These pressures are known to fluctuate with clinical status in CHF patients, and they provide key indicators for adjusting treatment regimens. For example, increases in enddiastolic pressure, changes in the characteristics of pressure within the diastolic portion of the pressure waveform, and decreases in maximum dP/dt, or increases in minimum dP/dt together suggest a deteriorating cardiac status. As used herein, LV max dP/dt refers to the maximum rate of pressure development in the left ventricle. These measurements could be obtained either during clinic visits or from the patient at home, from the proposed device and stored for physician review. The physician can then promptly adjust treatment. In addition, the system 10 may assist in management of patients when newer forms of device therapy (e.g., multiple-site pacing, ventricular assist as a bridge to recovery, or implantable drugs pumps) are being considered.

It may be useful to automate or partially automate some level of interaction with the patient. For example, departures from prescribed limits or values for certain patient parameters may be noted automatically and brought to the attention of the physician or patient. The ability to automatically select deteriorating patients from the much larger pool of monitored patients may save practitioner time and improve patient care.

It is contemplated that the system 10 may create an exception report on a daily basis to create a list of patients requiring special follow-up or care. More specifically, the system 10 may interact with the patient directly and request additional monitoring or compliance with a specific health care regime. The limits which trigger the exception report may be under the control of the attending physician.

More specifically, information received in the clinic by the PDCS 70 from the HDCS 60 may be evaluated and triaged for follow-up by a medical practitioner. Following evaluation of the information received in physician's office or clinic, the system 10 may create an exception report that lists patients to be contacted for follow-up.

Patients at home are monitored using the ITD 20 and HDCS 60 which transmit key information to the PDCS 70 for patient management to the physicians office or clinic. Information received by the PDCS 70 at the physicians office is used to determine if the patient's status is satisfactory or whether an adjustment in diet or therapy is required in order to maintain the patient's health and to prevent worsening of status that may eventually lead to hospitalization. On a given day, only a small percentage of patients may present with a deteriorating condition and require follow-up by a health care practitioner. It therefore is advantageous to evaluate patient information automatically using an algorithm that identifies those patients that require follow-up and a potential change in therapy. Such an algorithm may identify patients that require follow-up by, for example, analyzing current data vs. preset limits determined by the physician (e.g. if LV EDP>15 mmHg, then trigger follow up), or analyzing the results of a mathematical model applied to a waveform or portion of a waveform such as the diastolic portion of the LV pressure signal.

Once those patients are identified, an exception report is created that can be used by the practitioner when contacting patients. The exception report may include patient name, contact information (e.g. telephone number), identify which vital signs indicate a deteriorating condition, and other vital signs that are pertinent to follow-up. It may also be useful to list other information such as past problems the patient has experienced and/or the last time the patient's vitals were out of range. The system 10 may also provide the capability to contact the patient directly and automatically via a communication channel such as a cell phone or the Internet. Such communication could provide a message indicating to the patient a change in therapeutic regimen based on the information that has been obtained.

Description of ITD, RSA and TU

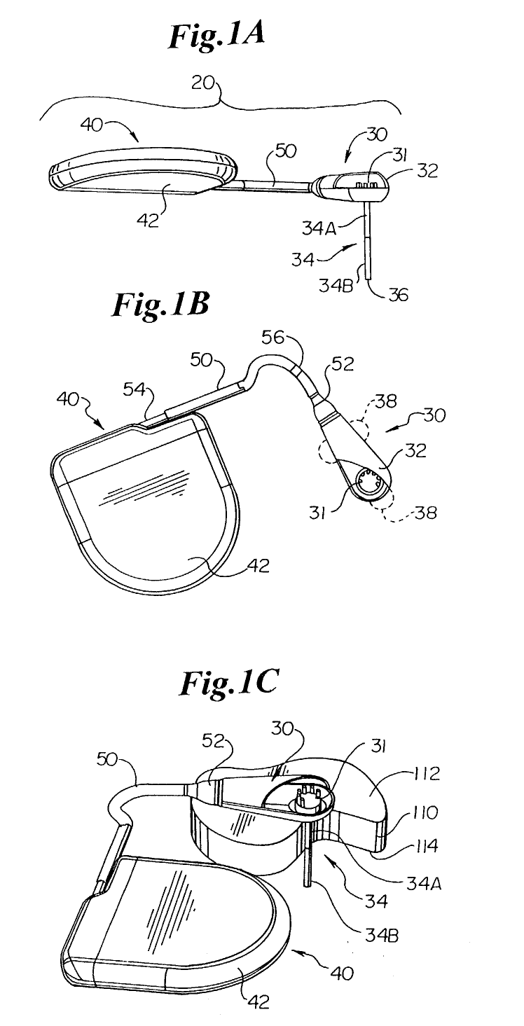

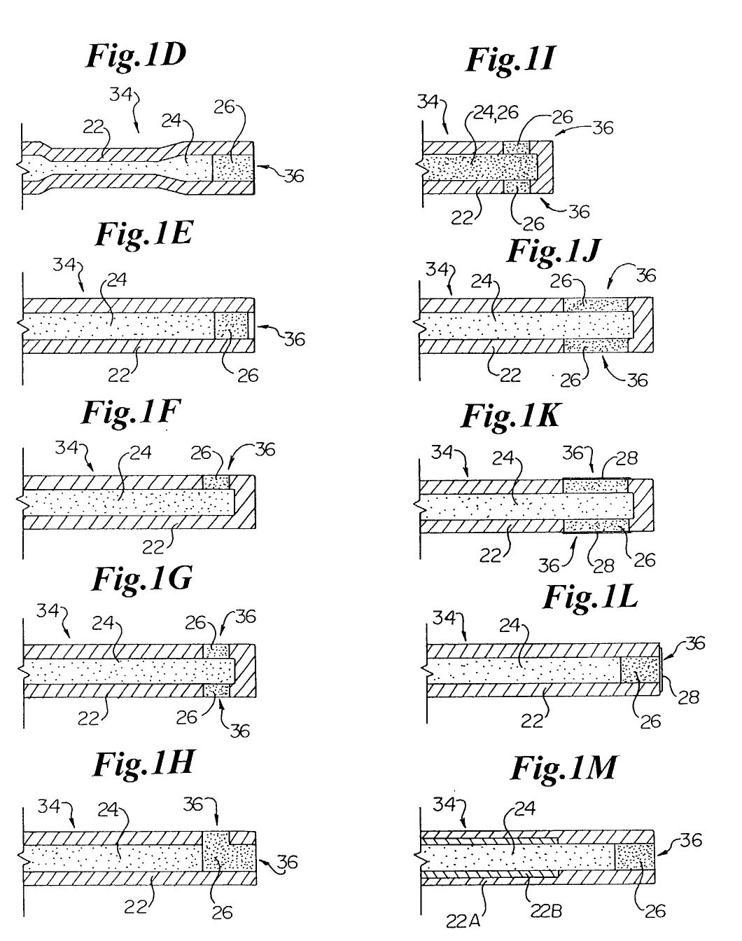

As mentioned previously, a system level explanation is provided with reference to FIGS. 8-14. Reference may be made to FIGS. 1A-1C to understand aspects associated with exemplary embodiments of the ITD 20, including the RSA 30 and the TU 40. Reference may also be made to U.S. Pat. No. 4,846,191 to Brockway et al., U.S. Pat. No. 6,033,366 to Brockway et al., U.S. Pat. No. 6,296,615 to Brockway et al., and PCT Publication WO 00/16686 to Brockway et al., the disclosures of which are hereby incorporated herein by reference, for examples of alternative embodiments of the RSA 30 and TU 40.

The RSA 30 includes a pressure transducer 31 and an electronics module 33 (not visible in FIGS. 1A-1C) contained within housing 32. The sensor housing 32 protects the pressure transducer 31 and the electronics module 33 from the harsh environment of the human body. The housing 32 may be fabricated of a suitable biocompatible material such as titanium and may be hermetically sealed. The outer surface of the housing 32 may serve as an electrogram (EGM) sensing electrode. The proximal end of the housing 32 includes an electrical feedthrough to facilitate connection of the electronics module in the housing 32 to a flexible lead 50. The distal bottom side of the housing includes a pressure transducer header to facilitate mounting of the pressure transducer 31 and to facilitate connection to a pressure transmission catheter (PTC) 34. The top side of the housing 32 may have a visible marking directly opposite the location of the PTC 34 on the bottom side such that the location of the PTC 34 can be visualized during surgery.

The housing 32 may include one or more connection means 38 such as suture rings (shown in phantom), tines, helical anchors, etc., to facilitate connection to tissue at the implant site (e.g., epicardium). As an alternative, the connection means 38 may comprise a mesh fabric (not shown) disposed over the housing 32 or integrally formed/connected to the bottom surface of the housing 32. Such a mesh fabric may be attached to the epicardial surface by adhesive, sutures, or other suitable means conventional in the surgical art. Enhanced long term attachment to the epicardial surface is enhanced by fibrosis which is encouraged by the rough texture of the mesh fabric. The mesh fabric may comprise a biodegradable or biodegradable/non-biodegradable material composite, and the outside (parietal) surface may be made smooth to minimize in-growth adhesion to the surrounding tissues (e.g., rib cage). Preferably the mesh fabric may have a partially open weave and/or be formed of a transparent or semi-transparent material to increase visibility through the mesh fabric reducing the likelihood of attachment to essential cardiac features.

The pressure transducer 31 and the electronics module 33 (not visible in FIGS. 1A-1C) disposed in housing 32 may be the same or similar to those described in U.S. Pat. Nos. 4,846,191, 6,033,366, 6,296,615 or PCT Publication WO 00/16686, all to Brockway et al. The electronics module 33 provides excitation to the pressure transducer 31, amplifies the pressure and EGM signals, and may digitally code the pressure and EGM information for communication to the telemetry unit 40 via the flexible connecting lead 50. The electronics module 33 may also provide for temperature compensation of the pressure transducer 31 and provide a calibrated pressure signal. Although not specifically shown, it may be useful to include a temperature measurement device within the electronic module to compensate the pressure signal from temperature variations. For example, the temperature measurement may select a look up table value to modify the pressure reading. This operation may be performed in any of the RSA 30, TU 40, or HDCS 60.

The PTC 34 refers pressure from the pressure measurement site (e.g., LV) to the pressure transducer 31 located inside the sensor housing 32. Various embodiments of the PTC 34 are illustrated in FIGS. 1D-1M. The PTC 34 may comprise a tubular structure 22 including a proximal shaft portion 34A and a distal shaft portion 34B, with a liquid-filled lumen 24 extending therethrough to a distal opening or port 36. The PTC 34 may optionally include one or more EGM electrodes or other physiological sensors as described in U.S. Pat. No. 6,296,615 to Brockway et al.

The proximal end of the PTC 34 is connected to the pressure transducer 31 via a nipple tube (not visible), thus establishing a fluid path from the pressure transducer 31 to the distal end of the PTC 34. The proximal end of the PTC 34 may include an interlocking feature to secure the PTC 34 to the nipple tube of the pressure transducer 31. For example, the nipple tube may have a knurled surface, raised rings or grooves, etc., and the proximal end of the PTC 34 may include an outer clamp, a silicone band, a spring coil or a shape memory metal (e.g., shape memory NiTi) ring to provide compression onto the nipple tube.

A barrier 26 such as a plug and/or membrane is disposed in the opening 36 to isolate the liquid-filled lumen 24 of the PTC 34 from bodily fluids, without impeding pressure transmission therethrough. If a gel (viscoelastic) plug 26 is utilized, one to several millimeters of a gel maybe be positioned into the opening 36 at the distal end of the PTC 34. The gel plug 26 comes into contact with blood and transfers pressure changes in the blood allowing changes in blood pressure to be transmitted through the fluid-filled lumen 24 of the PTC 34 and measured by the pressure transducer 31. The gel plug 26 is confined in the opening 36 at the tip of the PTC 34 by the cohesive and adhesive properties of the gel and the interface with catheter materials. The chemistry of the gel plug 26 is chosen to minimize the escape of the fluid in the remainder of the PTC 34 by permeating through the gel. In one embodiment, the fluid is chosen to be fluorinated silicone oil and the gel is chosen to be dimethyl silicone gel. Preferably, in order to inject the gel plug 26 into the opening 36 at the tip of PTC 34, as well as to obtain accurate measurements, the gel 26 may have a high penetration value. Penetration value is a measure of the “softness” of the gel by assessing the penetration of a weighted cone into the gel in a specified time. Also preferably, to meet in-vivo performance requirements for measuring blood pressure, the gel 26 may be soft enough to not induce hysteresis, but not so soft that significant washout occurs. Washout is also reduced by choosing a gel the becomes fully cross linked and has a low solubility fraction. The use of a fully cross-linked gel reduces if not eliminates permeation between the transmission fluid in lumen 24 and the gel material 26. Furthermore, a fully cross-linked gel is very stable, and thereby increases the usable life of the device.

The gel plug 26 may be flush with the distal end of the PTC 34 or may be recessed (e.g., 0.5 mm) to shelter the gel plug 26 from physical contact and subsequent disruption that may occur during the procedure of insertion into the heart. The undergelling may be achieved by way of stem compression during gelling to reduce lumen volume during filling or by thermally induced techniques. The gel barrier may be made flush with the distal end of the PTC 34 utilizing automated guillotine that cleanly and accurately severs the gel plug 26 distal to the tip. The distal tip of the PTC 34 and the gel plug 26 contained in the opening 36 may be protected by other means, such as by the use of a twist-on-off cap that mechanically interlocks on the proximal portion of the PTC providing protection of the tip/gel via an annular clearance. The tip protector may, in another embodiment, have a side release mechanism similar to a binder clip that again provides annular clearance at the tip but allows radial removal of the protector rather than axial. This annular clearance zone is more likely to be maintained during removal with the side clip approach. Either approach results in a cover which may be removed prior to insertion. Protection of the distal tip may also be achieved by utilizing a pocket defined in the final packaging that has sufficient clearance such that contact with the distal tip of the PTC 34 is avoided.

The pressure transmission fluid contained within the lumen 24 of the PTC 34 proximal of the barrier 26 may comprise a relatively low viscosity fluid and may be used to tune the frequency response of the PTC 34 by adjusting the viscosity of the transmission fluid. Preferably, the pressure transmission fluid comprises a relatively stable and heavy molecular weight fluid. Also preferably, the specific gravity of the transmission fluid is low in order to minimize the effects of fluid head pressure that could result as the orientation of the PTC 34 changes relative to the sensor 31. The pressure transmission fluid preferably has minimal biological activity (in case of catheter or barrier failure), has a low thermal coefficient of expansion, is insoluble in barrier 26, has a low specific gravity, has a negligible rate of migration through the walls of PTC 34, and has a low viscosity at body temperature. In one embodiment the pressure transmission fluid may incorporate end-group modifications (such as found in flourinated silicone oil) to make the transmission fluid impermeable in the barrier material 26. In another embodiment the fluid comprises a perfluorocarbon. Examples of suitable gels and transmission fluids are disclosed in U.S. Pat. No. 6,296,615 to Brockway et al.

With reference to FIGS. 1D-1M, various embodiments of the PTC 34 are shown in longitudinal cross section. In FIG. 1D, the PTC 34 comprises a tube 22 defining a fluid-filled lumen 24 therein extending to a distal facing port 36 having a gel plug 26 disposed therein. In FIG. 1D the gel plug 26 is flush with the distal end of the PTC 34, whereas in FIG. 1E, the gel plug 26 is slightly recessed from the distal end of the PTC 34.

In the embodiment shown in FIGS. 1A-1E, the opening 36 is located at the distal end of the distal shaft portion 34B, but may also be located in a side wall of the distal portion 34B as discussed with reference to FIGS. 1F-1K. In FIG. 1F, the PTC 34 includes a lateral facing port 36 filled with barrier material 26. In FIG. 1G, two lateral ports are provided, and in FIG. 1H, a single lateral port in combination with a distal facing port 36 are provided on the PTC 34.

The port(s) 36 may have the same cross-sectional area as the fluid-filled lumen 24, or the port(s) 36 may have a larger surface area (i.e., flared) than the lumen 24 of the PTC 34. A flared opening 36 reduces movement of the plug 26 during events that change either the volume of the transmission fluid or the internal volume of lumen 24, such as occurs during thermal expansion and contraction, bending, and hydration of the catheter material of PTC 34. Reducing the degree of displacement of plug 26 during bending of PTC 34 has the effect of reducing measurement artifact that can occur during normal movement of the subject into which the RSA 30 is implanted. Reducing the degree of displacement of plug 26 during bending of PTC 34 reduces the maximum amount of dead space (space defined by recessed plug 26 as seen in FIG. 1E) within PTC 34 and beyond plug 26, and therefore, contributes to improved patency in blood. Moreover, the larger surface area of the opening(s) 36 also increases the frequency response of the device.

As seen in FIG. 1D, the proximal and distal ends of the PTC 34 may be flared to have a larger inside diameter (ID) and outside diameter (OD), for different purposes. The distal end of the PTC 34 may be flared to provide an opening 36 having a larger surface area as discussed above, and the proximal end of the PTC 34 may be flared to accommodate the nipple tube (not shown) and provide a compression fit thereon. The proximal flared portion may have an ID that is smaller than the nipple tube to provide a compression fit that will be stable for the life of the RSA 30.

The mid portion or stem of the PTC 34 may have a smaller ID/OD, with gradual transitions between the stem and the flared ends. The gradual transitions in diameter provide gradual transitions in stiffness to thereby avoid stress concentration points, in addition to providing a more gradual funneling of the gel into the stem in the event of thermal retraction. The unitary one-piece construction of the PTC 34 may also provide a more robust and reliable construction than multiple piece constructions. Absent the gradual transitions, the PTC 34 may be more susceptible to stress concentration points, and the gel and the transmission fluid are more likely to become intermixed and may potentially dampen pressure transmission.

By way of example, not limitation, the proximal flared portion may have an ID of 0.026 inches, an OD of 0.055 inches, and a length of about 7 mm. The stem (mid) portion may have an ID of 0.015 inches, and OD of 0.045 inches, and a length of about 7 mm. The distal flared portion may have an ID of 0.035 inches, an OD of 0.055 inches, and a length of about 4 to 5 mm. The proximal taper may have a length of about 0.5 mm and the distal taper may have a length of about 1.25 mm. The gel plug 26 may have a length of about 3 mm and resides in the distal flared portion.

In cases where a relatively short PTC 34 is utilized, the fluid-filled lumen 24 of the PTC 34 may be completely filled with the barrier material 26 (e.g., gel). In combination with the gel plug 26, or in place thereof, a thin membrane 28 may be disposed over the port(s) 36. For example, as shown in FIG. 1K, a thin membrane material 28 is disposed over the lateral ports 36. As shown in FIG. 1L, a thin membrane material 28 is disposed over the distal facing opening 36. The thin membrane material 28 may comprise a thin, biocompatible polymeric material.

The PTC 34 should have a length sufficient to extend across a myocardial wall and into a heart chamber. For example, the proximal shaft portion 34A may have a length of about 10-15 mm, and the distal shaft portion 34B may have a length of about 2-15 mm. The PTC 34 preferably has a length that provides adequate access across the myocardium and into the left ventricle while being as short as possible to minimize head height effects associated with the fluid-filled lumen 24. The PTC 34 may be straight or may be curved, depending on the particular orientation of the RSA 30 relative to the heart wall and the chamber defined therein at the insertion point. An antithrombogenic coating may be applied to the distal portion 34B, and the proximal portion 34A may be overmolded with silicone to provide stress relief, flex fatigue strength, and a compliance matching mechanism at the entrance to the myocardium.

The PTC 34 may be positioned across a heart wall, with the proximal portion 34A extending across myocardium 110 and the distal portion 34B disposed endocardially, as schematically shown in FIG. 1C, and as will be described in more detail hereinafter. The proximal portion extends across the entire myocardial wall 110, from the exterior myocardial surface or epicardium 112, to the interior myocardial surface 114. Optionally, the proximal portion 34A may extend across the pericardium, epicardium and myocardium. Because the heart walls are dynamic structures subject to expansion and contraction, the proximal portion 34A may be made relatively crush-resistant, with sufficient crush resistance to prevent collapse caused by myocardial contraction. The distal portion 34B may be made relatively flexible with radiused corners to provide an atraumatic tip.

For example, as seen in FIG. 1M, the PTC 34 may comprise a stainless steel or titanium hypotube 22B (e.g., an extension of the nipple tube) extending through the proximal (myocardial) portion 34A, with a polymeric tube 22A extending over and beyond the hypotube 22B into the distal (endocardial) portion 34B. Alternatively, the proximal portion 34A may be formed of a polymeric material having a relatively high durometer and the distal portion 34B may be formed of a polymeric material having a relatively low durometer. The proximal and distal portions 34A/34B may be formed of separate tubes connected together, or by a single tube with a gradient stiffness, such as provided by interrupted layer coextrusion processes. As a further alternative, the proximal portion 34A and the distal portion 34B may comprise a polymeric tube having a relatively low durometer, with a rigid polymeric sleeve having a relatively high durometer extending over the proximal portion 34A.

The flexible lead 50 connects the electronics module 33 and sensor housing 32 to the telemetry unit 40. The lead 50 may contain, for example, four conductors—one each for power, ground, control in, and data out. The lead 50 may incorporate conventional lead design aspects as used in the field of pacing and implantable defibrillator leads. The lead 50 may include a strain relief 52 at the connection to the proximal end of the sensor housing 32. The lead 50 may also include a connector 54 which allows the RSA 30 to be connected and disconnected from the TU 40 in the surgical suite to facilitate ease of implantation, at a later time should it be necessary to change the TU 40, or for any other circumstance. The lead 50 may optionally include one or more EGM electrodes 56. When EGM electrodes are carried along the lead, the number of conductors will need to be modified to suit the design.

The TU 40 includes telemetry electronics (not visible) contained within housing 42. The TU housing 42 protects the telemetry electronics from the harsh environment of the human body. The housing 42 may be fabricated of a suitable biocompatible material such as titanium or ceramic and is hermetically sealed. The outer surface of the housing 42 may serve as an EGM sensing electrode. If a non-conductive material such as ceramic is used for the housing 42, conductive electrodes may be attached to the surface thereof to serve as EGM sensing electrodes. The housing 42 is coupled to the lead 50 via connector 54, and includes an electrical feedthrough to facilitate connection of the telemetry electronics to the connector 54. The telemetry electronics (not visible) disposed in the TU 40 may be the same or similar to those described in U.S. Pat. Nos. 4,846,191, 6,033,366, 6,296,615 or PCT Publication WO 00/16686, all to Brockway et al., and are discussed in more detail hereinafter.

Description of Transmyocardial Implant

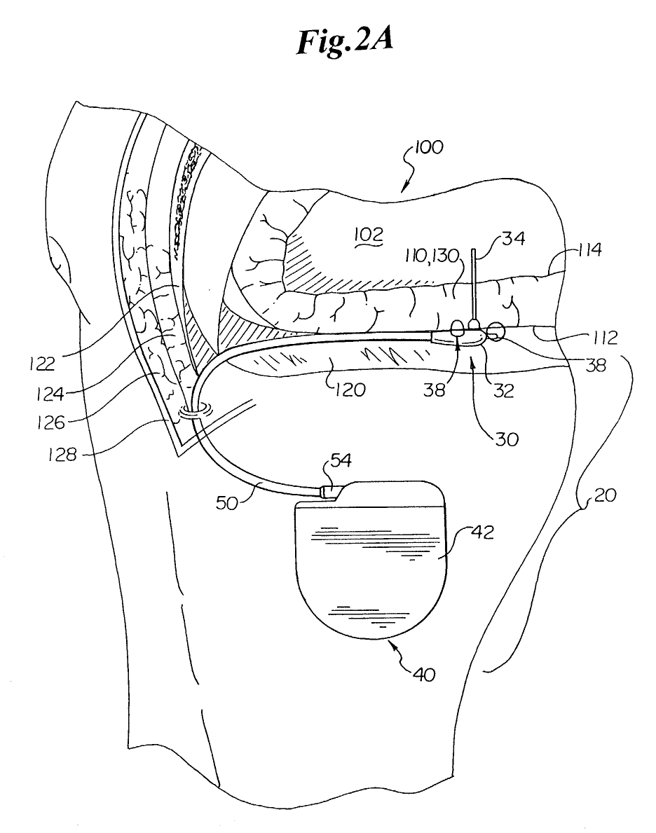

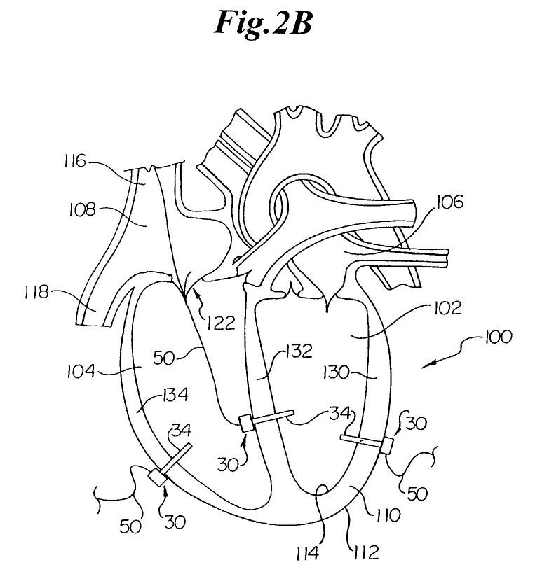

Refer now to FIG. 2A which shows the ITD 30 surgically implanted in/on a heart 100 of a patient. In this exemplary embodiment, the present invention provides for insertion of the PTC 30 directly into the LV 102 across the wall 130 (i.e., myocardium 110) 110 of the heart 100 for the purpose of measuring LV pressure. This allows for chronic monitoring of pressure in the LV chamber 102 of the heart 100.

Implantation of the ITD 20, including RSA 30 and TU 40, may take place during an open chest procedure such as would normally be done to perform coronary artery bypass or valve repair/replacement. Alternatively, the ITD 20 may be implanted in a separate surgical procedure. In such a case, the surgeon performs a median sternotomy, cutting across the dermal layer 128, sub-dermal tissue 126, muscle layer 124, and sternum 122. The surgeon then cuts the pericardial sac 120 to expose the heart 10, down to the LV apex.

The PTC 34 is introduced into the LV 102 at the inferior apical segment using a split-sheath introducer (not shown). The split-sheath introducer facilitates insertion of the PTC 34 into the myocardium 110 and protects the PTC 34 from damage that may otherwise occur during the insertion process. Following insertion of the PTC 34, the split-sheath introducer is removed and discarded.

The split sheath introducer may incorporate handles that extend outward beyond the periphery of the RSA 30 for easy access. The handles may be relatively long with raised ears of softer durometer to facilitate easy griping. A solid core needle (trocar) may also be used to eliminate coring and emboli formation which may be associated with hollow needles and to maximize compression exerted on the PTC 34 by the myocardium, thereby accelerating hemostasis. Radiopaque materials may be incorporated into the PTC 34, the split sheath introducer (not shown), and/or the trocar to insure trackability via x-ray fluoroscopy.

The PTC 34 is automatically positioned within the LV 102, in terms of depth, by virtue of its length when the housing 32 of the RSA 30 contacts the myocardial surface. In other embodiments wherein the length of the PTC 34 and the housing 32 do not limit depth penetration, the PTC 34 may be positioned within the LV chamber 102 by pulling the PTC 34 back until the pressure signal disappears, and then advancing the PTC 34 approximately 2-10 mm to assure that the tip is not in the immediate proximity of trabeculae (not shown). Inserting the PTC 34 as such reduces the likelihood that fibrous tissue will overgrow the tip of the PTC 34. The entry point of the PTC into the epicardium 112 may be secured for hemostasis by fine purse string suture. The purse string sutures may extend through the epicardium and into the myocardium. The sensor housing 32 may then be anchored to the pericardium with a fine suture material utilizing the suture ports 38 integrated into the sensor housing 32. The sensor housing 32 and PTC 34 are positioned in a manner that provides sufficient slack in the portion of the PTC 34 external to the myocardium 110 in order to absorb stress. Again, these steps are useful with embodiments wherein the length of the PTC 34 and the housing 32 do not limit depth penetration into the LV chamber 102. The embodiment illustrated in FIG. 2A does not require these particular steps for correctly positioning the PTC 34 in the LV chamber 102.

Turning back to the specific embodiment illustrated in FIG. 2A, the proximal lead 50 is then draped over the open pericardial edge, and brought caudally inferior laterally under the abdominal fascia. A 4-5 cm horizontal incision is made on the left upper quadrant of the abdominal wall and a subcutaneous pocket is created. The proximal end of the flexible lead 50 may be brought into the subcutaneous pocket through an introducer placed through the abdominal fascia. If a releasable connection 54 is utilized, the lead 50 is attached to the TU 40, tested using a PDCS, and the TU 40 is placed in the subcutaneous pocket. The pocket and the chest are then closed.

Refer now to FIG. 2B which illustrates various possible anatomical implant positions for the RSA 30. To facilitate a discussion of the various possible anatomical implant positions, the heart 100 is shown schematically. The heart 100 includes four chambers, including the left ventricle (LV) 102, the right ventricle (RV) 104, the left atrium (LA) 106, and the right atrium (RA) 108. The LV 102 is defined in part by LV wall 130, RV 104 is defined in part by RV wall 134, and the LV 102 and the RV 104 are separated by septal wall 132.

The right atrium 108 receives oxygen deprived blood returning from the venous vasculature through the superior vena cava 116 and inferior vena cava 118. The right atrium 108 pumps blood into the right ventricle 104 through tricuspid valve 122. The right ventricle 104 pumps blood through the pulmonary valve and into the pulmonary artery which carries the blood to the lungs. After receiving oxygen in the lungs, the blood is returned to the left atrium 106 through the pulmonary veins. The left atrium 106 pumps oxygenated blood through the mitral valve and into the left ventricle 102. The oxygenated blood in the left ventricle 102 is then pumped through the aortic valve, into the aorta, and throughout the body via the arterial vasculature.

By way of example, not limitation, the RSA 30 may be implanted such that the distal end of the PTC 34 resides in the LV 102, the RV 104, or any other chamber of the heart 100, although the LV 102 is preferred for the reasons set forth previously. For example, the PTC 34 may be positioned across the LV wall 130 such that the distal end of the PTC 34 is disposed in the LV 102 as described with reference to FIG. 2A. Alternatively, the PTC 34 may be positioned across the RV wall 134 such that the distal end of the PTC 34 is disposed in the RV 104 in a similar manner as that described with reference to FIG. 2A. If the ITD 20 comprises a unitary structure containing both the RSA 30 and the TU 40, the ITD 20 may be entirely positioned within a heart chamber. As a further alternative, the PTC 34 may be positioned across the septum 132 separating the LV 102 and the RV 104.

Description of Transeptal Approach

In this later embodiment, the RSA 30 may be translumenally delivered into the RV 104, with the PTC extending across the septal heart wall 132 such that the distal end of the PTC 34 is disposed in the LV 102. In particular, a minimally invasive catheterization procedure may be employed to deliver the RSA 30 to the ventricular septum 132 via the superior vena cava 116, RA 108, tricuspid valve 122 and RV 104. The TU 40 may be placed in the pectoral region with the lead 50 extending from the RSA 30 along the delivery path to the TU 40. Alternatively, the TU 40 may be integral with the RSA 30 and reside within the RV 104 as described in PCT Publication WO 00/16686 to Brockway et al.

A suitable delivery method for the transeptal approach is to deliver a transeptal catheter and needle percutaneously via the superior vena cava under local anesthesia. Single or biplanar radiographic techniques may be used to visualize the catheter and needle during the procedure. The catheter and needle are advanced until the distal end of the catheter is adjacent the septum 132 in the RV 104. The needle is advanced within the catheter until it punctures through the septum 132. The catheter is then advanced over the needle until the distal end of the catheter is disposed in the LV 102. The needle is then removed from the catheter leaving the catheter in place to define an access path across the septum 132 via the lumen in the catheter.

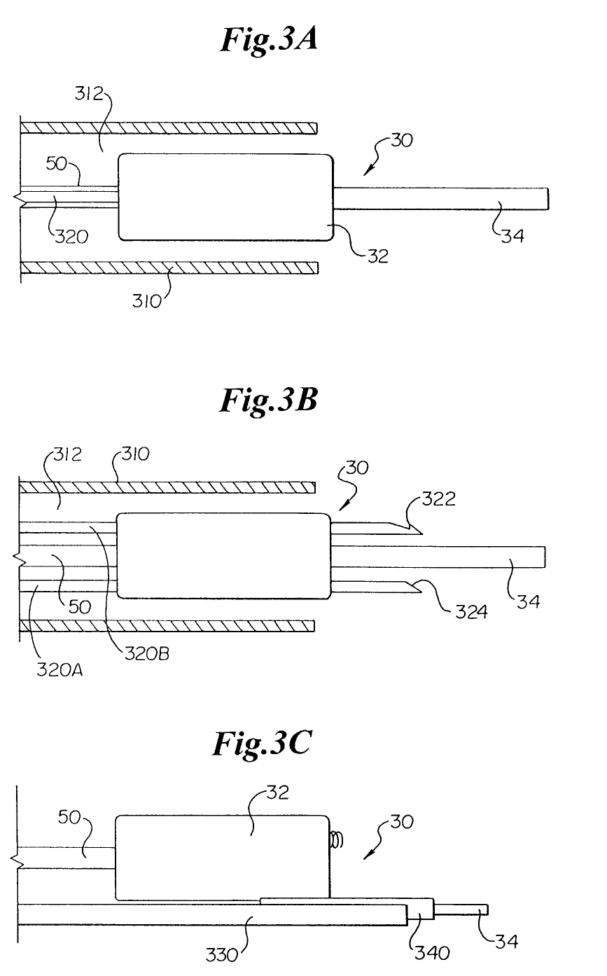

Refer now to FIGS. 3-7 which illustrate various devices and design aspects that may be employed to translumenally deliver the RSA 30 as described above. With specific reference to FIG. 3A, translumenal delivery may be facilitated with a delivery catheter 310 inserted into the RV 104 utilizing conventional techniques. The distal portion of the delivery catheter 310 may be curved to allow for positioning of the distal end adjacent the septal wall 132.

The RSA 30 may be advanced through the lumen 312 of the catheter 310 using a guide wire or push rod 320 until the distal tip of the PTC 34 engages the septal wall 132 on the RV 104 side. The lead 50 may also be used to advance the RSA 30 through the catheter 310, and may incorporate a stiffening rod to increase column strength. The guide wire or push rod 320 may terminate within the housing 32 of the RSA 30 as shown or extend through the housing 32 parallel and exterior to the PTC 34. To facilitate smooth delivery through the catheter 310 and proper alignment with the septal wall 132, the RSA 30 and PTC may be coaxially aligned and cylindrically shaped as shown.

The RSA may then be advanced further such that the PTC 34 is inserted into and across the septal wall 132 until the distal end resides within the LV 102. The housing 32 may then be secured to the septal wall 132 utilizing barbs, cork-screw, tines, helical wires, threads possibly in combination with shape memory effects or the like. The delivery catheter 310 and guide wire 320 may then be withdrawn leaving the RSA 30 in place with the PTC 34 extending across the septal wall 132 into the LV 102, and with the lead 50 extending along the return delivery path. The proximal end of the lead 50 may then be connected to the TU 40, and the TU 40 may be implanted in a suitable location such as a subcutaneous pocket. Alternatively, the TU 40 may be integral with the RSA 30 and reside within the RV 104 as described in PCT Publication WO 00/16686 to Brockway et al.

As seen in FIG. 3A, guide wire or push rod 320 may be concentrically positioned relative to the RSA 30 and reside in a guide wire lumen within the RSA 30 and/or the lead 50. Alternatively, as seen in FIG. 3B, two guide wires 320A/320B may be employed, each eccentrically positioned relative to the RSA 30 and residing within a guide wire lumen extending through the RSA 30. The guidewires 320A/320B assist in advancing the PTC 34 through the septal wall 132. To this end, the guide wires 320A/320B may include a barbed tip 322 or a sharpened tip 324 to penetrate and cross the septal tissue. In addition, the guide wire lumens in the RSA 30 may include a oneway mechanism such that pushing on the guide wires 320A/320B causes distal advancement of the RSA 30, and pulling on the guide wires 320A/320B does not cause displacement of the RSA 30.

As a further alternative, the PTC 34 may reside within a tube 340 attached to the housing 32 as seen in FIG. 3C. The guide wire tube may have a circular profile and reside within a slotted delivery catheter 330. The slotted delivery catheter 330 may be similar to a conventional coronary guide catheter, with the addition of a slot extending along at least a distal portion thereof. The slot in the delivery catheter 330 may have a width that is less than the diameter of the tube 340 such that the tube 340 slides within the catheter 330 but does not fall out. The delivery catheter 330 may be used to protect the PTC 34 during delivery and puncture through the septum or heart wall. The delivery catheter 330 may have a sharp tip to facilitate puncture of the septum or heart wall. With this arrangement, the RSA 30 may be advanced along the catheter 330 to the implant site, and the delivery catheter 330 may be withdrawn leaving the RSA 30.

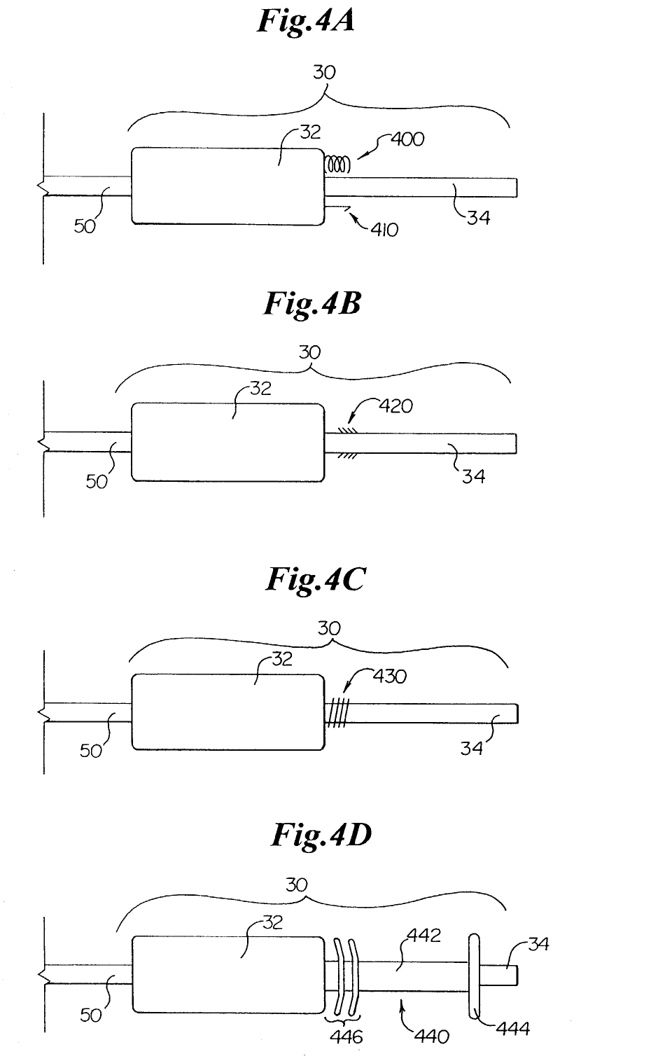

As mentioned previously, the housing 32 of the RSA 30 may be anchored to the septal wall 132 on the RV 104 side using an anchor means such as cork-screw 400 or barbs/tines 410 as shown in FIG. 4A, rearward-facing ridges or flanges 420 as shown in FIG. 4B, or helical threads 430 as shown in FIG. 4C. The anchor means may be connected to the housing 32 as shown in FIG. 4A, or to the PTC 34 as shown in FIGS. 4B and 4C.

As a further alternative, the anchor means may comprise a somewhat flexible spool-shaped structure 440 disposed on the PTC 34 as shown in FIG. 4D. The spool anchor 440 may be fixedly connected to the PTC 34 or include a connection means such as a snap-fit or internal threads. The spool anchor 440 includes barrel portion 442 which extends across a hole in the septal wall 132. The hole may be formed using a puncture device such as guide wire 320 with a sharpened tip 324, and preferably has a diameter slightly larger than the barrel portion 442 of the spool anchor 440. The spool anchor also includes a distal rim 444 which is disposed in the LV 102 against the septal wall 132 and has a diameter which is larger than the hole in the septum. The distal rim 444 may be delivered across the hole in the septum through a catheter which temporarily dilates the septal hole. Alternatively, the distal rim 444 may be hinged, similar to proximal rims 446. Proximal rims 446 are similar to distal rim 444, and may be hinged to allow collapse through the septal hole. Utilizing multiple proximal rims 446 accommodates septal walls of different thickness.

If the distal tip of the PTC 34 is used to puncture the septal wall 132, it is desirable to utilize a distal portion 34B that has sufficient column strength to avoid buckling when crossing the septal wall 132. As such, the relatively rigid and crush resistant proximal portion 34A discussed with reference to FIGS. 1A-1C may be extended into the distal portion 34B. In addition, to avoid coring septal tissue as the distal tip of the PTC 34 punctures the septal wall 132, a dissolvable material 510 such as Manitol may be disposed in or around the opening 36 distal of the membrane or gel barrier disposed therein. Once implanted, the material 510 dissolves and pressure communication is reestablished with the opening 36.

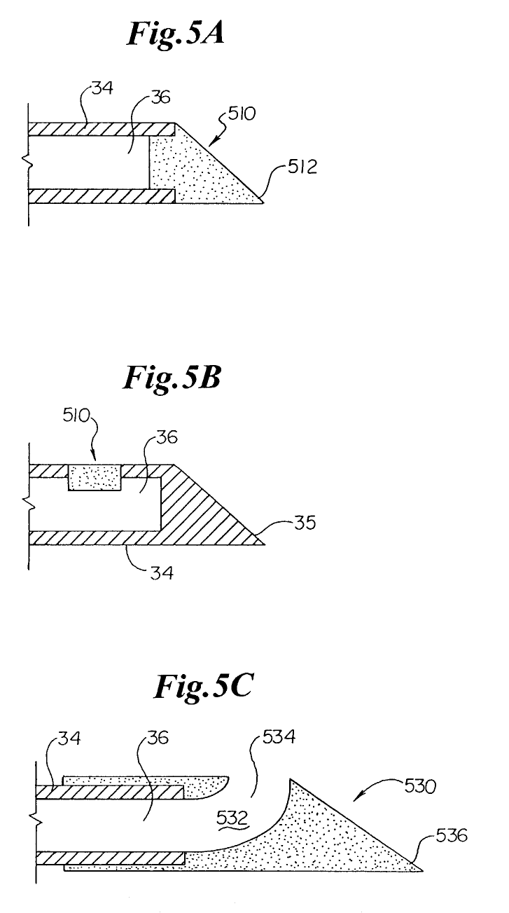

Various configurations are possible with such a dissolvable material 510. For example, as seen in FIG. 5A, dissolvable material 510 may be disposed in opening 36 at the distal end of the PTC 34, and may define a sharpened tip 512 to ease puncturing the septal wall 132. As seen in FIG. 5B, the dissolvable material 510 may be disposed in opening 36 located in the side of PTC 34. In this latter embodiment, the distal tip of the PTC 34 may include a sharpened tip 35 to facilitate puncturing the septal wall 132.

As yet another alternative to avoid coring, a special tip 530 may be attached or formed onto the distal end of the PTC 34 as seen in FIG. 5C. The tip 530 may be formed of a dissolvable material such as Manitol, and may define a lumen 532 which provides a pressure referring path between the distal-facing opening 36 of the PTC 34 and the lateral-facing opening 534 of the tip 530. Because the opening 534 of the tip 530 faces laterally, the likelihood of coring septal tissue is reduced if not eliminated.

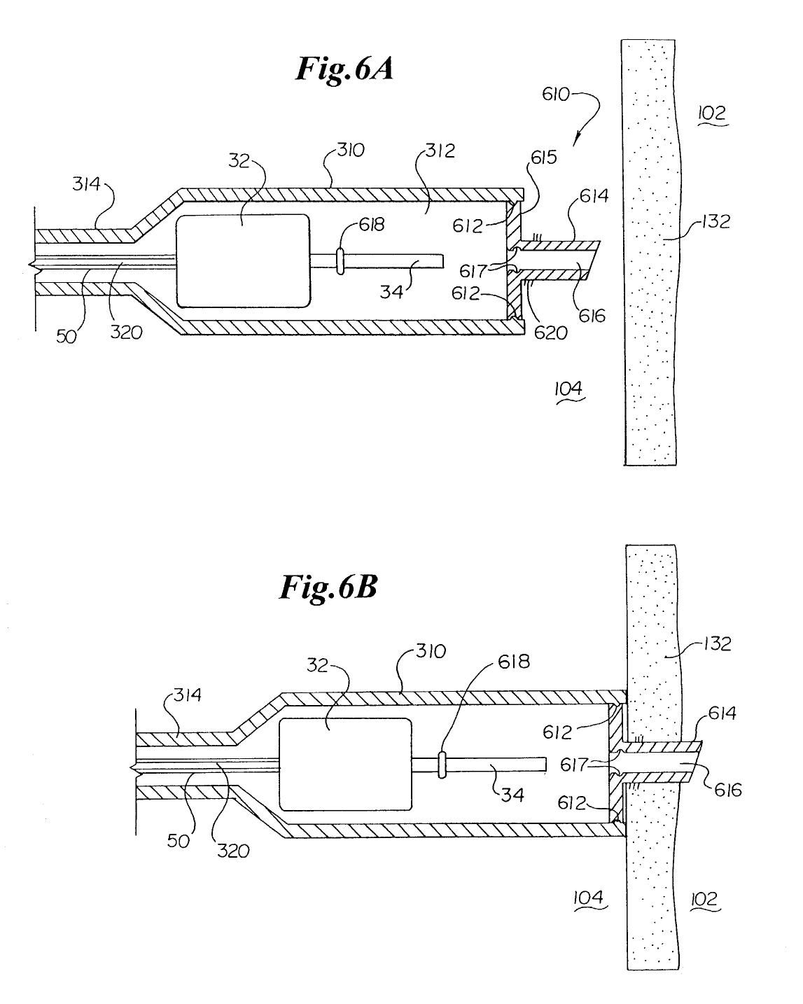

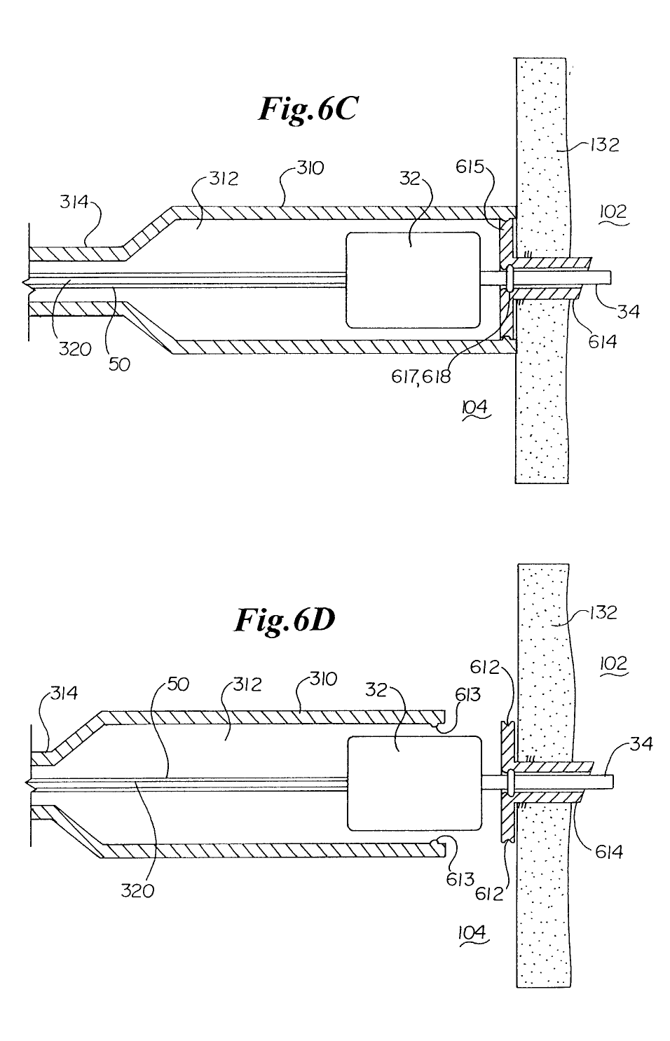

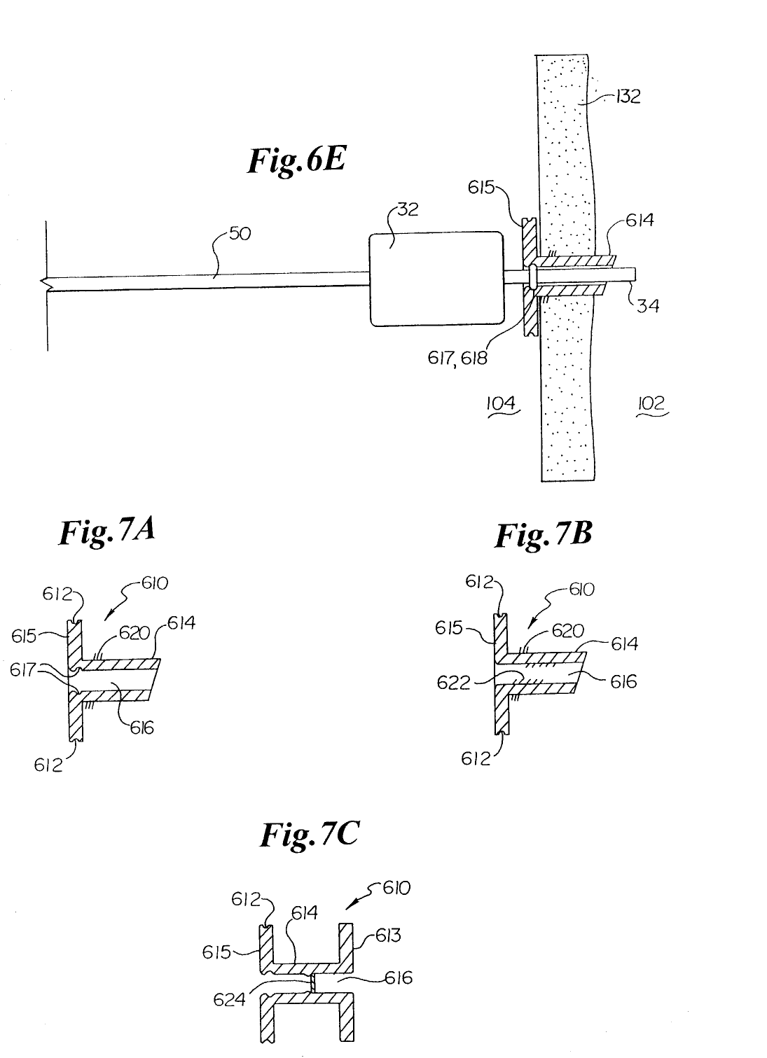

Refer now to FIGS. 6A-6E which schematically illustrate a method of delivering the PTC 34 across the septal wall 132, similar to the method described with reference to FIG. 3A, but utilizing a different anchor means. In this particular embodiment, the RSA 30 is connected to the septal wall 132 utilizing anchor device 610. Anchor device 610 includes a body portion 614 and a proximal flange 615, which is releasably attached to the delivery catheter 310 by connector 612. Connector 612 may comprise a wide variety of releasable mechanisms such as mating threads or a mating snap-fit geometry (nub and recess) 612/613 as best seen in FIG. 6D.

Body portion 614 includes a sharpened tip to penetrate septal tissue, and is sized to traverse the septal wall 132. Anchor members 620 are disposed on the body portion 614 for anchoring to septal tissue. Anchor members 620 may comprise barbs, tines, cork-screw, threads, etc. Body portion 614 also includes a lumen 616 that is sized to accommodate the PTC 34 therein, and a connector 617/618 for releasable connection to the PTC 34. Connector 617/618 may comprise a wide variety of releasable mechanisms such as mating threads or a mating geometry (nub 618 and recess 617) as shown.

In use, the catheter 310 is navigated to the RV 104 as discussed with reference to FIG. 3A and shown in FIG. 6A. The catheter 310 may be preloaded with the anchor device 610 and optionally preloaded with the RSA 30, and is advanced until the tip of the body portion 614 is adjacent the septal wall 132 as shown in FIG. 6A. The catheter 310 is then pushed distally and rotated if necessary to cause the body portion 614 of the anchor device 610 to penetrate the septal wall 132, with the anchor members 620 engaging the septal tissue as shown in FIG. 6B.

The delivery catheter 310 protects the PTC 34 of the RSA 30 during delivery, and may optionally be sharpened or otherwise include means to assist the PTC 34 across the septal wall 132. For example, the delivery catheter 310 may include a balloon or other expandable structure to provide back-up support against the RV wall 134 opposite the septum 132. In addition or in the alternative, a vacuum may be applied to the lumen of the catheter 310 such that the catheter 310 is temporarily anchored to the septum 132 when the distal end thereof engages the septum. In addition or in the alternative, mechanical means (hooks, tines, screws, etc.) may be used to grasp the septal wall 132.

With the body portion 614 extending across the septal wall 132 and into the LV 102, the RSA 30 is advanced in the catheter 310 by pushing on lead 50 and/or guide wire/push rod 320 until the releasable connector 617/618 engages between the anchor device 610 and the PTC 34 as shown in FIG. 6C. The releasable catheter 310 is then retracted in the proximal direction until the releasable connector 612/613 disengages the catheter 310 from the anchor device 610 as shown in FIG. 6D. The catheter 310 and guide wire/push rod 320 are then removed leaving the RSA 30 and anchor device 610 in place, with the PTC 34 extending across the septal wall 132 and into the LV 102 as shown in FIG. 6E.

The catheter 310 may have a constant diameter as shown in FIG. 3A, or a conforming profile with a reduced diameter distal portion (not shown). If a catheter 310 having a reduced diameter distal portion is used, the RSA 30 may be advanced in the catheter 310 after the catheter 310 is in place, or the RSA 30 may be preloaded into the catheter 310, and the catheter 310 and the RSA 30 may be advanced together. The RSA may include distal blades (not shown) mounted on the distal end of the housing 32 to facilitate removal of the catheter 310 from over or around the RSA 30 by pulling the catheter 310 proximally and slitting the distal reduced diameter portion of the catheter 310 with the blades.

As mentioned previously, various releasable connection mechanisms may be used to connect the anchor device 610 to the PTC 34. For example, as discussed with reference to FIGS. 6A- 6E, and as shown in detail in FIG. 7A, the releasable connection mechanism may comprise a mating snap-fit geometry. Alternatively, as shown in detail in FIG. 7B, the releasable connection mechanism may comprise threads 622 which mate with threads disposed on the PTC 34.

With any of these embodiments, the anchor device 610 may be initially connected to the catheter 310 for puncturing and traversing the septal wall 132, or the anchor device 610 may be initially connected to the PTC 34 (with or without catheter 310) to support the PTC 34 as it punctures and traverses the septal wall 132. As yet a further alternative, the anchor device 610 may be permanently implanted into the septal wall 132 to facilitate repeated access across the septal wall 132.

Such a permanent anchor device 610 may incorporate radiopaque markers or materials to facilitate easy, accurate and repeatable access across the septum 132. To facilitate permanent implantation, a proximal flange 615 and a distal flange 613 may be used, and it may be delivered using Mullin's technique, for example, similar to the embodiment discussed with reference to FIG. 4D.

To prevent cross-flow of blood between the LV 102 and the RV 104 across the septal wall 132, such an anchor device 610 may incorporate a valve 624 disposed in the lumen 616 as shown in FIG. 7C.

The foregoing discussion with reference to FIGS. 1-7 has focused on the ITD 20 (i.e., RSA 30 and TU 40) and its delivery to various sites in/on a patient's heart 100. The following discussion with reference to FIGS. 8-14 focuses on the functional aspects of the system 10.

Overview of System Electronics

FIG. 8 depicts the overall system architecture 10 and this figure is presented to facilitate a discussion of the exemplary and preferred partitioning and location of the electronic components. It should be understood that other configurations and partitioning are acceptable and within the scope of the invention.

The implanted portion of the system 10 comprises telemetry device (ITD) 20, which includes the remote sensor assembly (RSA) 30 and the telemetry unit (TU) 40, connected by an implantable lead 50. The implanted portion of the system 10 communicates with a remote station called a home data collection system (HDCS) 60.

The HDCS 60 may comprise, for example, a wearable monitor or a unit that is placed in the patient's home and interacts with the ITD 20. In this context, the HDCS 60 may be a local communication base station for the ITD 20. The HDCS 60 is described in more detail elsewhere in the specification.

The HDCS 60 periodically sends data to a physician data collection system (PDCS) 70. The PDCS 70 may receive data from more than one patient. Typically, the PDCS 70 may be a dedicated office type computer operating with specialized software to aid a physician in evaluating the patient's condition by storing and analyzing patient data over time. The PDCS 70 is described in more detail elsewhere in the specification.

Barometric Pressure Correction

The accuracy of the pressure measured by the implanted pressure transducer 31 is influenced by external pressure changes (i.e., barometric pressure) and is preferably corrected to avoid inaccuracies and/or possible misinterpretation of pressure data. Barometric pressure can change significantly when a weather front moves through the area where the patient resides, when the patient is riding up an elevator in a tall building or traveling in mountainous areas where changes in elevation are frequent and significant. Thus, the present invention provides a number of different pressure correction schemes as described herein. Those skilled in the art will recognize that the correction methods described herein are applicable to a wide variety of parameters that may be measured by an implantable transducer and which require correction by measurements obtained from an external reference measurement.

Although specific correction schemes are described in detail elsewhere in the specification for purposes of illustration, not limitation, a brief description of some general approaches is provided below. One general approach is to take barometric pressure measurements simultaneously with measurements taken by pressure transducer 31, and subtract the barometric reading from the internal pressure measurement. For example, the HDCS 60 may take a barometric pressure reading and subtract the barometric pressure measurement from the pressure measurement transmitted by TU 40 of the ITD 20.

In some situations, it is desirable to record pressure measurements within the ITD 20. This can eliminate the need to transmit data at frequent intervals, thereby reducing power consumption of the ITD 20. There are a number of such pressure data correction and storing techniques that may be employed with ITD 20.

In a first approach utilizing a data storage technique, pressure data may be stored in memory within the ITD 20 and then transmitted occasionally to an external device such as HDCS 60. A local barometric pressure recorder, which may be incorporated into the HDCS 60, records measurements at preprogrammed intervals. The HDCS 60 then pairs measurements from the ITD 20 and the barometric pressure recorder in time and then make a correction based on the pair. Further details of this approach are described in U.S. Pat. No. 5,810,735 to Halperin et. al., the entire disclosure of which is hereby incorporated by reference.

In a second approach utilizing a data storage technique, pressure data may be transmitted from an external barometric pressure measurement device, which may be incorporated into the HDCS 60, to the ITD 20. The ITD 20 then corrects the in vivo pressure measurement with the value of the barometric pressure communicated from the external device. The corrected value is stored in memory within the ITD 20. The corrected values are then wirelessly transferred at a later time to the HDCS 60. Further details of this approach are described in U.S. Pat. No. 5,810,735 to Halperin et. al. and U.S. Pat. No. 5,904,708 to Goedeke, the entire disclosures of which are hereby incorporated by reference.

In a third approach utilizing a data storage technique, a barometric pressure monitor (BPM) is located external to the body, and measures barometric pressure at times specified by a controller. Measurements obtained by the BPM are representative of the barometric pressure to which the body of the patient is exposed. The BPM may be a small device attached to a belt, worn on the neck as a pendant, on the wrist like a watch, or placed in a purse or briefcase. The BPM may be incorporated into the HDCS 60, for example.

At some time, e.g. the first measurement obtained after the BPM is powered on, the absolute value of barometric pressure is stored in the memory of a computing device, which may be incorporated into the BPM, for example. The absolute value of barometric pressure is stored in the memory along with a time stamp (e.g. year, month, day, hour, minute and second). From then on, each subsequent barometric pressure measurement is compared to the stored measurement and evaluated to determine if the difference between that measurement and the stored measurement exceeds a predetermined threshold (e.g. 0.5 mmHg). If the difference is less than the threshold, no further action is taken on that measurement. If the difference is greater than or equal to the threshold, then that value is saved in memory along with a time stamp. If a chronic time series is collected from the patient, the memory of the computing device in the BPM contains barometric pressure values at each point in time where the pressure changed significantly (significant as determined by the preset value).

With this third approach, pressure measurements taken by the ITD 20 are made with respect to a specific reference pressure, normally to a vacuum. Pressure measurements are recorded into memory in the ITD 20. Measurements are stored in a way that allows the date and time of the recording to be established. At various times, the pressure measurements recorded in the ITD 20 are transferred to an external combining device (CD) through means of a wireless link. The CD may also be incorporated into the HDCS 60, for example, and the BPM also has the ability to transfer measurements to the CD. This transfer can be made through a hard link (e.g., electrically conductive wires or fiber optics) if the BPM and CD are in the same unit such as HDCS 60, or via a wireless link (e.g., RF transmission) if the BPM and CD are remote from each other. Once data from both the ITD 20 and the BPM are transferred to the CD, the CD can correct the measurements obtained from the ITD 20 for barometric pressure. Knowing the barometric pressure measurements at the starting time and at each point thereafter when pressure changes by a significant amount, it is possible to know the barometric pressure at any time up until the date and time of the last value recorded in memory. Correction of a measurement from the ITD 20 for barometric pressure can be achieved by subtracting the barometric pressure measurement reconstructed at that time point, or by mathematically manipulating the two time series to achieve a result equivalent to subtraction.

A variation of this third approach is to record corrected measurements within the ITD 20. In some cases it is useful to have the corrected pressure measurements available within the ITD 20, such as when the ITD 20 is in communication with a device that is providing therapeutic effect, such as an infusion pump, pacemaker or defibrillator, and is relying on accurate pressure measurements to adjust the therapy parameters. Such a therapeutic device may be implanted or external (e.g., a drug infusion pump or wearable defibrillator)

The BPM may transmit barometric pressure data to the ITD 20, which subtracts the barometric measurement from the in vivo pressure measurement and utilizes or otherwise stores the corrected measurement. Alternatively, the in vivo pressure measurements may be transmitted to the BPM which corrects the pressure measurement from the ITD 20 for barometric pressure and transmits the corrected pressure measurement back into the ITD 20.

Alternately, the BPM may evaluate the barometric pressure measurements as they are obtained. In this alternative embodiment, the BPM would transmit the barometric pressure to the ITD 20 when it is first turned on or brought into the receiving range of the BPM. Once this initial measurement is received by the ITD 20, if a measurement differs from the previous value by more than a predetermined threshold, then (and only then) would the BPM transmit a barometric pressure measurement to the ITD 20. The ITD 20 would then send a confirming transmission to the BPM indicating that the transmission of barometric pressure was correctly received. The BPM may continue to send the measurement at regular internals until such confirmation is received.

Incorporation of Additional Sensors

An example of the use of this device 10 is for the monitoring of endocardial pressure and the exemplary discussion is directed toward this application. However, it is anticipated that pressure transduction sensors may be combined with other transducers to provide a more complete assessment of cardiac function.

Temperature

It is contemplated that a temperature measurement device may be placed in the RSA 30, such as on the electronics module 33, to measure temperatures typically in the range of 35-42 C. The temperature measurements may be telemetered out of the patient as an independent measurement for the HDCS 60 or PDCS 70 to use to correct the pressure measurement for errors due to temperature variations. The temperature data may also be used internally in the ITD 20 to calibrate and compensate the pressure transducer for variations in pressure measurement due to temperature changes.

Ultrasound

It is contemplated that a small ultrasonic transducer may be placed on the myocardial side of the RSA 30 in contact with the heart wall. Acoustic signals may be transmitted and reflection time may be measured to determine the thickness of the heart wall or the inside dimension of the heart chamber along the vector viewed by the ultrasonic transducer. These parameters may be monitored over time to follow the patient's condition.

Impedance

It is contemplated that the PTC 34 may be used to carry two or more electrodes into the heart wall. A frequency signal may be applied to two electrodes which induce a signal on the remaining electrodes as a function of the tissue impedance. Tissue impedance may be monitored with the system to measure the condition of the heart.

Biopotential Sensors

It is contemplated that one or more biopotential sensing electrodes may be incorporated into the RSA 30 to monitor localized electrical activity in the myocardium. Analysis of the chaotic nature of the localized electrical signals within the myocardium may provide information that would be useful in assessing impending rhythm disturbances such as tachyarrhythmia or fibrillation.

Oxygen

It is contemplated that an oxygen sensor may be placed on the PTC 34 to reside in the wall of the heart or in the heart chamber to measure oxygen saturation of the blood or tissue. Chemical, electrochemical and optical sensors are contemplated for this application. It is anticipated that the value of oxygen is a useful metric for assessing the clinical status of heart failure patients.

Respiratory/Stroke Volume

It is contemplated that one or more additional electrodes may be incorporated into the lead 50 to measure respiratory effort and/or stroke volume. For example, one electrode may be provided on the lead 50 to measure EGM, and a second electrode may be provided at the other end of the lead 50. A constant current carrier signal may be applied across the electrodes. Respiratory changes and stroke volume changes cause impedance changes across the electrodes and may be detected by amplitude modulation of the carrier signal. The amplitude modulated signal may be demodulated and band-pass tailored for respiratory signals producing a changing voltage proportional to respiratory effort. Cardiac stroke volume may be obtained using similar techniques but with a band-pass tailored to the cardiac signal.

Activity

It is contemplated that a sensor to monitor physical activity of the patient may be provided in the TU 40. Activity may be a useful metric in combination with other parameters such as LVP in assessing the status of heart failure patients.

Overview of ITD Structure

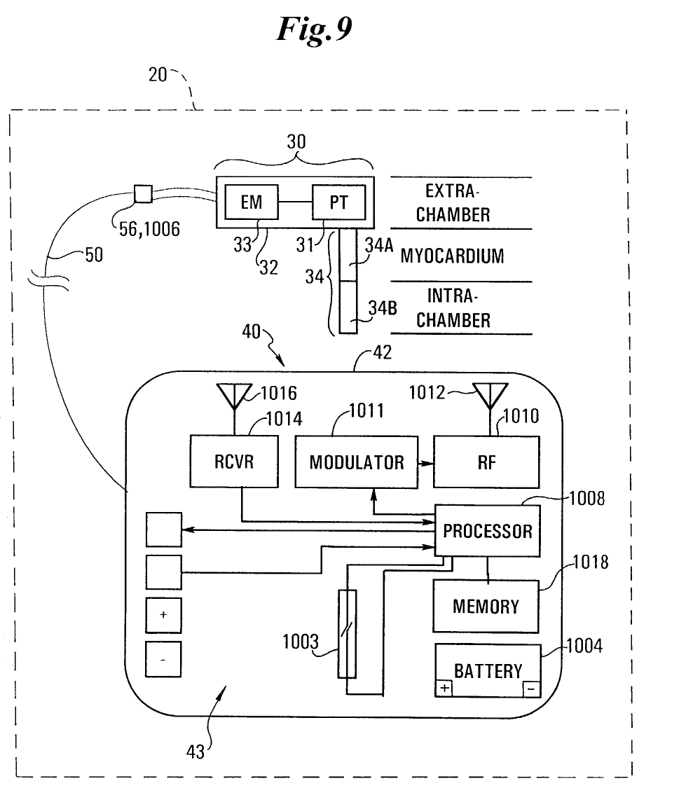

FIG. 9 is a more detailed schematic representation of the architecture of the implantable telemetry device (ITD) 20. The partitioning is preferred, but alternative architectures and modifications will be apparent to one of ordinary skill in this art.

The telemetry electronics module 43 in the TU 40 provides excitation to the pressure transducer 31 and sensor electronics module 33 in the RSA 30. The sensor electronics module 33 amplifies the pressure and EGM signals, and digitally codes the pressure and EGM information for communication to the TU 40 via the flexible connecting lead 50. The sensor electronics module 33 may also provide for temperature compensation of the pressure transducer 31 and provide a calibrated pressure signal that is identical for each catheter, allowing for complete interchangeability of the RSA 30 among TUs 40. For example, the temperature measurement may select a look up table value to modify the pressure reading. The look up table values may be derived from temperature and pressure measurements taken during or after the time of manufacture.

The sensor electronics module 33 may be used to avoid noise issues with communication of the pressure signal from the sensor to the TU 40. By amplifying the signals near the distal end of the catheter and converting them to a digital serial bit stream or pulse position modulated pulse train to communicate to the TU 40, errors due to supply voltage drop with lead resistance, noise induced by magnetic fields, stray capacitance, and leakage currents from penetration of body fluids into the flexible lead are avoided. This approach also simplifies the design of the connector, allowing a standard connector from the pacing/implantable defibrillator industry to be used.

The flexible lead 50 that connects the RSA 30 to the TU 40 may, for example, contain four conductors—one each for power, ground, control in, and data out. In one embodiment, the lead includes standard materials and technology from the pacing and implantable defibrillator lead industry. To facilitate easy removal from the patient, the lead may be isodiametric with the sensor housing and has surface characteristics that reduce friction with the fribrotic tissue that will grow around the lead.

The lead 50 may in some embodiments, carry one or more electrodes typified by electrode 1006. In some embodiments these lead mounted electrodes may be used to sense the depolarization of the heart. It is also possible to sense depolarization between the housing 32 of the RSA 30 and the housing 42 of the TU 40. Unipolar and bipolar sensing are possible and the selection of optimal electrode areas will follow normal industry practice.

In one embodiment of the ITD 20, the microprocessor 1008 keeps track of timeof-day and turns on the RF oscillator/transmitter 1010 on a schedule which may be once a day at a specific time. The modulator 1011 keys the transmitter 1010 to send out pressure data in real time through antenna 1012. More complex systems are possible as well. For example, the electrodes 1006 may be coupled to an EGM rhythm analysis module to collect and format heart rhythm data which is sent from the RSA 30 to the TU 40. This rhythm information may be sent from the TU 40 to the HDCS 60.

It must be recognized that heart rhythm data may also be extracted from the pressure time history as well as from the electrode sites. It may also be desirable to collect rate data from both the electrode sites and the pressure time histories to compare measured rates for calibration and diagnostic purposes.

It may also be desirable to measure heart rate and to couple the pressure data with the heart rate data. It may be useful to “bin” or correlate the pressure data based upon an average heart rate over a short interval. The binning or short term averaging process may be used as an alternative to a time of day protocol. The use of heart rate to qualify the pressure data ensures that the patient is in the same cardiac state during each transmission. In this example, the heart rate is used as a proxy for the cardiac or physiologic state of the patient.

Some communication protocols may require that the telemetry device TU 40 be “bidirectional” and upload data as well. The tuned receiver 1014 and associated antenna 1016 may cooperate with the microprocessor 1008 to reprogram configuration data in memory 1018.

In use, the RSA 30 may be mounted on the wall of the beating heart, while the TU 40 is implanted under the skin in the patient's chest or abdomen, somewhat remote from the RSA 30 and the heart. Both the RSA 30 and TU 40 may be hermetically sealed to protect the electronic components. Mounting the RSA 30 on the heart wall places the PTC 34 in the heart which places the pressure transducer 31 close to the heart as well. By locating the pressure transducer 31 near the surface of the heart, the pressure sensing is largely independent of posture because it greatly reduces the static head pressure artifact.

The ITD includes a battery 1004 which provides periodic transmissions (e.g., 30 second to 8 minute at hour intervals) over the specified in vivo operating life. The terminals of a battery may be bridged with a capacitor to regulate current draw. A rechargeable cell may be placed in parallel with the primary cell to power the electronics. In practice, the charging time for the rechargeable cell may be monitored to measure primary cell capacity or “life”. It should also be understood that the motion of the body may be transduced into power by a piezoelectric element or the like to power the electronic components.

The ITD 20 may include means for indicating to the physician at the time of implantation that the remaining battery capacity will support the specified continuous use battery life. The unit may indicate the remaining useful life with an accuracy of 20% at any time following implantation.

The ITD 20 may have an in vivo operating pressure range between −25 to 300 mmHg gauge pressure and range of ambient pressures are pressures equivalent to those encountered from sea level to 8,000 feet above sea level (ASL).

Blood pressure may be measured as a gauge pressure (relative to barometric pressure). However barometric pressure is not readily available in within the body. It is proposed that in some embodiments the pressure transducer 31 of the RSA 30 may be a differential type transducer with the reference port of the transducer connected to or in communication with the thoracic cavity. Thus, in this alternate embodiment, differential pressure is measured rather than absolute pressure. The differential is between an endocardial measurement and the reference thoracic measurement which is similar to barometric pressure.

The implanted pressure transducer 31 may be subject to long term drift and aging effects. Since the sensor itself is implanted, it is not available for direct replacement or recalibration. It is proposed to monitor an easily measured system pressure as a proxy, and to correlate this measured pressure with a transducer pressure under the same conditions. One method would be to compare LV systolic pressure with aortic systolic pressure. Periodically, the clinical measurement is made and the sensor reading are compared to compensate for drift in the implanted sensor. In general, the internal conversion parameters stored in the ITD may be altered via telemetry to render permanent the changes in calibration.

Another alternative to long term calibration is the use of a low drift secondary sensor with very stable trigger pressure. When the secondary sensor indicates that this trigger pressure is reached, the system instantaneously calibrates the primary pressure sensor to the trigger pressure. The secondary sensor may be exposed to the pressure in a peripheral vessel which may be monitored while the calibration is occurring to verify the integrity of the calibration process.

In another embodiment, a non-invasive measurement of central aortic systolic pressure may be used to recalibrate the LV pressure measurements. Devices capable of accurately measuring central aortic pressure non-invasively are known in the art.

In various embodiments, the ITD 20 includes different operating modes. For instance, in a bidirectional system the HDCS 60 may send a signal to the ITD 20 telling it to transmit data, the ITD 20 transmits information for 0 to 8 minutes following activation. The HDCS 60 can also send a signal to the ITD 20 to instruct it to stop transmitting. In the alternative, the ITD 20 and HDCS 60 may turn on at a particular time of day and the ITD 20 would transmit real time data that the HDCS 60 transfers in real time to the more remote PDCS 70. A magnet 1003 may be provided to force the TU 40 to enter or exit a data transmission mode.

An example of a modulation scheme is pulse position modulation where the location of a pulse or burst in between two framing pulses is preferred for the simplicity of implementation. However, such analog schemes are susceptible to “jitter”. It is proposed to place a frequency shift within the burst to designate the pulse. The FSK encoding methodology is also less variable as the range between the transmitter in the ITD 20 and the HDCS 60 vary.

Overview of HDCS Structure

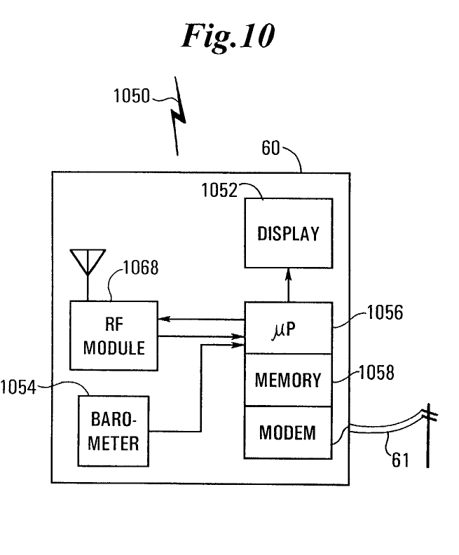

Turning to FIG. 10, the HDCS 60 is described. The TU 40 and HDCS 60 communicate through a radio frequency telemetry link 1050 which is preferably unidirectional from the TU to the HDCS but bidirectional communication is contemplated as well. Although radio frequency communication is preferred other modalities are contemplated as well, including acoustic, galvanic conduction, and light transmission. The HDCS 60 may be located near the patient (e.g. bedside) or on the patient (e.g., worn on the patient's belt). The HDCS 60 may comprise a single unit, or two cooperative units, such as one unit being worn by the patient which communicates with another unit located nearby or some distance away.

The HDCS 60 is intended to provide short range communication with the ITD 20 and has a minimal user interface consisting primarily of a display 1052 which is used to notify the patient of successful operation of the system and more particularly to notify the patient of various error conditions.

Another important feature of the HDCS 60 is the presence of the ambient pressure monitor reference 1054 that is coupled to the microprocessor 1056. This ambient pressure monitor 1054 is used as a barometer to compensate for barometric pressure induced changes in the data telemetered from the implantable TU 40. Highly accurate and stable barometric references are expensive and require care to maintain their accuracy. For this reason it is anticipated that the pressure transducer in the HDCS 60 may require recalibration and it is anticipated that the calibration process may include comparison of the barometer monitor 1054 with the pressure reference contained in the PDCS 70. The PDCS 70 may send calibration data to the HDCS 60 to compensate for drift or other inaccuracies of the ambient pressure monitor 1054 response. In general, since barometric pressure varies widely with location the calibration requires that the HDCS 60 and PDCS 70 be at the same location.

In one embodiment of the system 10, the internally measured pressure is compensated for barometric (i.e., atmospheric) pressure at the bedside or when the pressure measurement is transmitted. However it may be desirable to have pressure data collected at a time when the PDCS 70 is not available. It is anticipated that the patient may wear or carry an barometric pressure monitor (BPM) and this device may either upload barometric data to the implanted device to compensate the cardiac pressure measurement or alternatively the implanted unit may down load endocardial pressure measurements to the BPM. An alternate data logging strategy may be used as well. If the endocardial data is time stamped and the barometric data is time stamped the two data sets may be combined. If the barometric pressure has not varied much during an endocardial data collection episode then the endocardial data may be used directly without multiple sequential compensations.

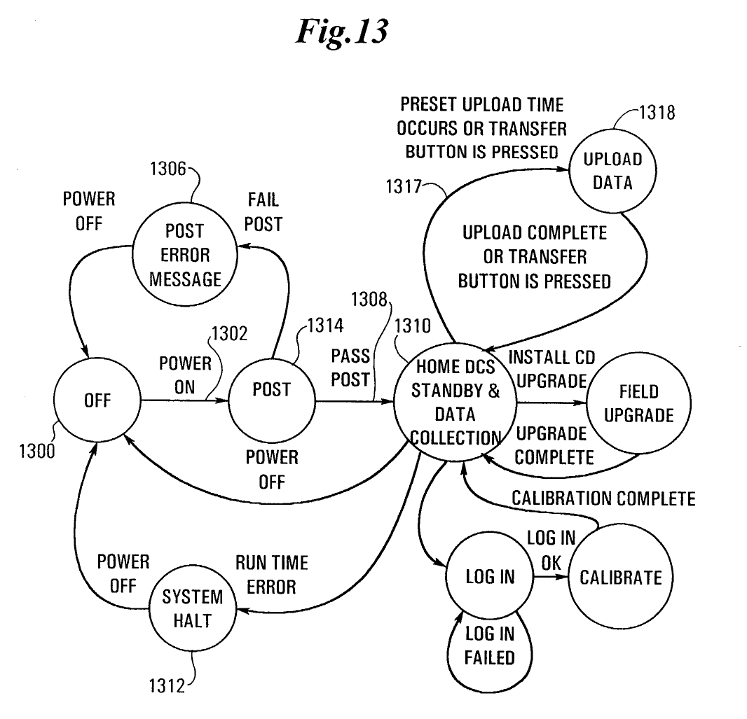

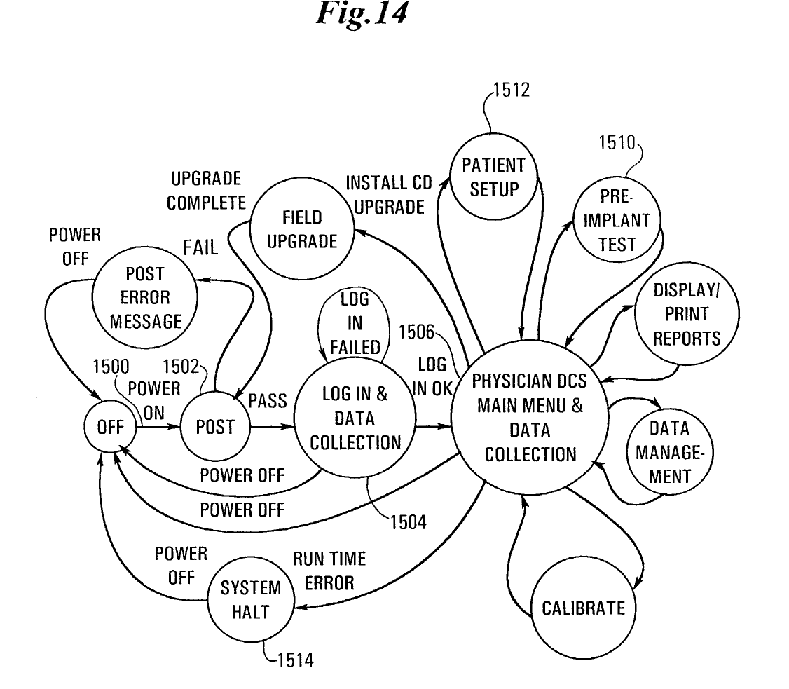

The data sent by the ITD 20 to the HDCS 60 may be stored locally at the HDCS 60 where it is time stamped and compensated for changes in ambient pressure. The microprocessor 1056 operates under the control of a program stored in memory 1058 as explained in connection with FIG. 13.

The patient data is sent to a remote physician data collection system 70 (PDCS) which is typically at the physician's office. The communication link 61 is preferably either a modem connection or connection through the Internet.

In one embodiment, a clock within the ITD 20 awakens the TU 40 and it transmits left ventricular pressure in real time to the local receiver 1068 in the HDCS 60. The HDCS 60 receives the pulse position modulated signal and compensates it for local barometric pressure and pressure variations, and the compensated data is stored in the HDCS 60 until it is transmitted to the PDSC 70. Thus, rather than being interrogated by the HDCS 60, the ITD 20 may be programmed to automatically transmit data to the HDCS 60, and such program may vary, for example, the interval between acquisitions of parameters, and/or the duration of the waveform from which parameters are derived.

In some embodiments, a mechanism may be provided to immediately turn off the TU 40 in the event that the TU 40 is interfering with other critical medical devices. For example, the TU 40 transmission may be terminated immediately by application of a magnet that may be incorporated into a device such as a wand that is always readily available.

In some embodiments, two resident transmitters/receivers may be provided in the TU 40 to allow data transmission and programming in the event of a failure of one of the transmitters/receivers. For example, the TU 40 may change transmission of the pressure data from a high frequency transmitter to a low frequency transmitter in the event of a failure of the high frequency transmitter. This would reduce the range of transmission but would allow data to be collected. The TU 40 may use the high frequency transmitter as part of the bi-directional programming sequence in the event of a failure of the low frequency transmitter. This would allow the TU 40 to still be programmed. In addition, a magnet activation sequence may be used to set an implant to default settings in the event of a failure of one of the implant receivers. This would allow data to be transmitted for a typical Pressure Measurement Transmission (PMT) schedule if the TU 40 receiver fails when the PMT schedule is too frequent, leading to premature battery failure, or when the PMT schedule is not frequent enough or turned off, resulting in a shortage of data.

The system may have the capability for bidirectional communications between the HDCS 60 and ITD 20. In this instance the RF section 1068 also has transmit functionality. The HDCS 60 controls the operation of the TU 40 based on a sampling protocol stored in the HDCS. Control signals are forwarded to the TU 40 via RF link 1050, instructing the TU 40 when to transmit data. When the TU 40 transmits data, the HDCS 60 is also capable of sending patient information to the TU 40 for storage and later retrieval. The sampling protocol is stored within the HDCS 60. The HDCS 60 is capable of acquiring data at regular intervals, or continuously. The most common sampling mode is for the HDCS to acquire a 1-minute waveform of LV pressure and EGM data, derive clinically relevant parameters from the waveforms (such as max+LV dP/dt), and store those parameters in the HDCS memory. It is also possible to store the waveforms in the HDCS memory and to make computations for the calculated parameters at the PDCS 70.

Other types of data processing that may be performed in the HDCS 60 may include: conversion of telemetered data to common units such as mmHg; removal of certain types of telemetry noise from the data; verification that the data received is from the ITD 20 implanted in the correct patient; verification that the signal is sufficiently free of noise that the parameters derived from the signal will be accurate within the specified acceptable tolerance ranges; and/or derivation of certain clinically relevant parameters from the telemetered signal.

The HDCS 60 will be capable of communicating the recorded data to the Internet. Communication of data from the HDCS 60 to the Internet can take place via a number of methods. The options include; connect the HDCS 60 to a personal computer via an interface cable (e.g. RS232, USB, etc.), infrared link, or RF telemetry link; connect directly to the Internet via a cell phone link; pass data from the HDCS 60 to a telephone connection located in the patients home via a dedicated device that interfaces to the phone line via a telemetry link from the HDCS 60 (e.g. bluetooth or other RF link).

As described above, the pressure data collection process takes a relatively high bandwidth real time analog signal from the ITD and transmits this ultimately to a remote physician user who collects data for many patients over a long period of time. As a consequence the total amount of data collected is very large and data compression will be useful to make the data set manageable. It has been estimated 4.4 terabytes of information are generated by this system for 10,000 patients in a two year period for each server. In addition to normal data compression techniques, it is proposed that certain data reduction techniques be practiced in a preferred embodiment.

The proposed process begins with the uncompressed waveform data which is transmitted in an analog fashion from the ITD to the HDCS. The analog waveform is typically pulse position modulated and it is digitized at the HDCS. The results of the A to D conversion of the analog waveform may be stored as an array of two byte integers. Pressure transducer measurements will range between −250 to 2500 as integer values. Standard commercially available compression techniques such as Huffman compression operating directly on the integer values have been tested on data sets resulting in a compression ratio in the range of 18 to 20%. It is proposed to not store or transmit the actual measurements but rather store the differences between sequential measurements. This simple “delta” process results in a reduction in the amount of data required to reconstruct the waveform. A further reduction of data can be achieved by applying the “delta” process to the reduced data set yielding a “delta delta” compression. In this instance the process gives a 90-96% compression ratio when used in conjunction with Huffman compression techniques .

It is possible to convert the delta delta data into a single byte stream of data for transmission to remote PDCS devices. To allow for outlying signal values it may be useful to select and designate certain values as indicative that the next transmitted value requires two bytes to encode rather than one byte. The so called exception code allows for high bandwidth but only when needed.

Overview of PDCS Structure

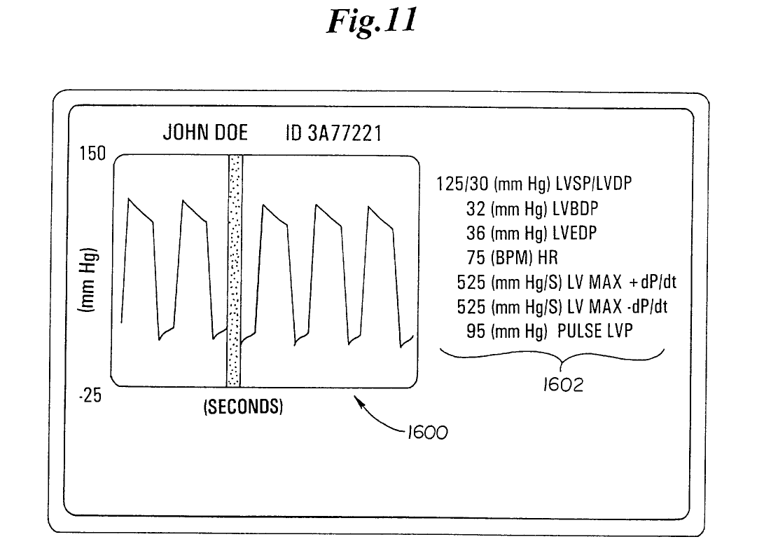

The PDCS 70 displays and permits time based analysis of a patient's pressure history. The physician uses the pressure trend data to assist in making a diagnosis of heart disease and most particularly congestive heart failure as described herein. The PDCS 70 may comprise, for example, a dedicated office type computer system. The display and analysis software may reside on the computer or a network. An example screen display is set forth as FIG. 11. Exemplary software control is explained in connection with FIG. 14.

In one embodiment, the physician's computer is used in health care facilities. It may be a non-dedicated PC running on an operating system platform commonly found in the physician's office. In one embodiment, the physician's computer is used in the clinic, OR, ER, hospital ward, and ICU. It interfaces to the HDCS 60 via the modem. The purpose of this is to provide a means of changing the protocol controls without having to go through the Internet. It also provides a means of viewing data from the HDCS in real time in a higher-resolution and more viewable format than what is available on the HDCS display 1052.

The PDCS 70 also provides a means for obtaining hard copy of data. In order for the physician to be reimbursed for a reading, Medicare requires that the medical record contain a 2-minute hardcopy strip. In addition, the physician will want to place a hardcopy of data in the medical record whenever the data is used to make a clinical decision.

If the PDCS 70 is implemented as an Internet browser application, then the Internet based software must perform several functions; such as create and manage a database of data collected from large numbers (tens of thousands, eventually the capacity for 100's of thousands) of patients; provide data reduction and viewing software (DRVS) to the physician upon request; download data from a specific patient upon receiving a physician request via the data reduction and viewing software; provide protocol control configuration software (PCCS) to the physician upon request; download protocol controls to the HDCS 60; manage access rights to data and settings; and manage database of device settings.

In one embodiment, when the physician wants to connect to the Internet to run the software, the software may be designed to assure that the software the physician will use is the most recent version. The system may provide access to all stored data only by authorized individuals. The integrity of the security of the data base is important. Access to data from a specific patient will by physician. Several physicians may have access to data from a given patient. Access to data must meet the requirements of medical record privacy laws in the U.S., Europe, and Japan. In one embodiment, the database (or portions thereof) can be used for research purposes, so it will be necessary to be able to partition the data to provide by clinical researchers to all or subsets of the stored data.

The system is designed to serve data to a number of physicians. The times indicated below assume that the physician has high-speed Internet access. From the time the physician clicks on the bookmark for access to the software, the log on screen is designed to appear with 5 seconds with a likelihood of about 80%. The logon is designed to be simple and quick, with a physician option to save the security access code. Once a security code is validated, the physician is only required to enter the patient name and within 5 seconds, the system validates that the physician has access to the data from that patient. A selection of templates associated with how the physician has previously viewed data from that patient is available. The template selection can include standard ways of viewing the data, or the physician can create a customized view. Once the desired template is selected, the physician need only to click on a button to instruct the Internet application or download data. Downloading of parameter data for a single patient for a 3-month period would occur within 20 seconds.

The PDCS 70 provides the means for the physician to view the data downloaded from the HDCS 60 in a number of different ways. In one embodiment, software performs cursory analysis of the data and provides one or more statistics to the user. For example, in FIG. 11, a sample template for viewing the data of a “John Doe” is shown. Both pressure trace 1600 and computed data 1602 is available for review, on a real-time or historical basis. Although there are a wide variety of diagnostic measurement that may be made with the system it is important to note that the patient may have a pacemaker or other rhythm based device present. It will be possible to optimize the pacing therapy for example based upon the pressure measurements made with the system.

There are numerous ways of presenting the measured data. Many physicians prefer two axis displays of pressure against time. However given the total complexity of the data it may be preferred to use polar representations of some data sets. For example a loop graph may be prepared where the polar angle represents the location in the cardiac cycle and the radius can represent absolute value of a measured variable such as pressure or dP/dT.

State Transition Descriptions

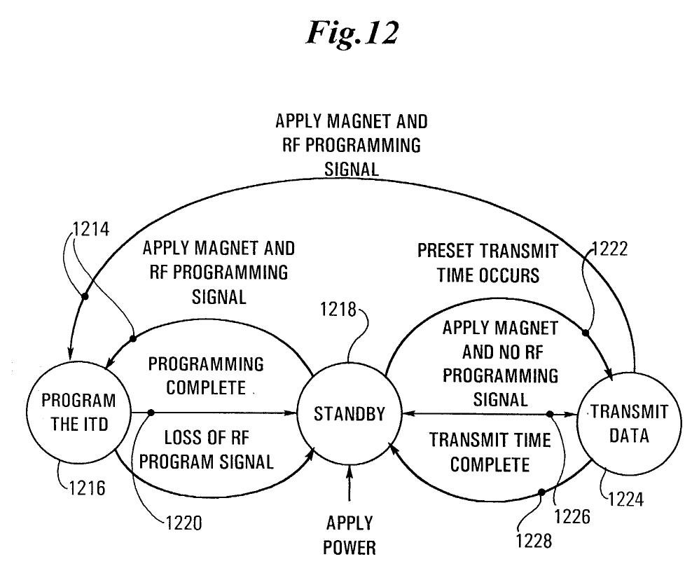

The state transition descriptions set forth in FIGS. 12-14 disclose the major operating states for the parts of the system 10. As further described elsewhere, each major part of the overall system incorporates a computer and operates under the control of a stored program. In most instances the operating firmware remains fixed for the life of the device but in field service upgrades may be made to the software to refine and extend operation of the devices.

Turning to FIG. 12, there is a state transition diagram for the implantable telemetry device 20. The device enters the standby state 1218 once power is applied. If a magnet is applied and an RF programming signal occurs 1214, the device moves to the program state 1216. In the program state the ITD is programmed with data necessary for it to carry out its implanted task. In general clock values are loaded into the device as well as transmission time settings. For example, the time for the device to begin transmissions is loaded while the length of transmission may be set from approximately 0 seconds to 2 minutes. Additionally, patient data identifying the ITD to a particular patient may be programmed as well. Once the device is completely configured it enters the standby state 1218 through state transition 1220. In standby state the device can transmit real time data at a specific time of day by entering the transmit data state 1224 through the state transition 1222. The device may also be forced to transmit data immediately by the application of a magnet through the apply magnet state transition 1226. At the completion of the data transmission cycle the device reenters the standby state 1218 through state transition 1228. As previously indicated, additional complexity may be developed in the ITD to permit bi-directional transmission. Although the preferred data-transmitting format is based upon the burst of RF, it should be appreciated that both pressure as well as temperature signals may be conveniently transmitted from the device.

Turning to FIG. 13, there is a state transition diagram for the local data collection system. The device leaves the off state 1300 by turning on power. The power on transition 1302 takes the device to a diagnostic power on self-test mode 1314. In this state various diagnostic tests are performed on the hardware and software. Assuming that the device fails the self-test it posts error messages in state 1306 and then reenters the off state 1300 by turning the power off. If the device passes the diagnostic tests, then state transition 1308 takes the device to the standby and data collection mode 1310. In this state, the device time stamps incoming data from the ITD 20 and stores it in the memory of the local data collection system. Run time errors detected will result in a system halt state 1312 and the device reenters the off state 1300 by turning power off. State transition 1317 takes the device into an upload data state 1318 where the HDCS 60 is capable of uploading information to the PDCS 70 via the Internet, for example. During state 1318, the device may communicate remotely with the PDCS 70 via the Internet, for example, to upload data directly from the HDCS 60 via time of day or manually by transfer button activation 1317.

The software operating in the local data collection system can be upgraded by entering into a field upgrade state initiated by selection after installation of a CD and a CD-ROM drive in the device. At the completion of the upgrade the CD-ROM is ejected from the drive.

Turning to FIG. 14, The PDCS 70 operates under the control of software and the simplified state diagram shows the various operating states of the preferred device. It should be understood that not all operations are required for a functioning system and various modifications can be made without departing from the scope of the invention.

When power is turned on as indicated by state transition 1500 the device enters the power on self-test state 1502. Assuming that the various self-test procedures are successfully completed the device prompts the user for password and name in state 1504. If the login is successful then the device displays the main menu in state 1506. A background process runs during state 1506. In operation the PDCS 70 may be downloading data from implanted ITDs from various locations. This data is episodic but is likely to be nearly real time when received.

It is possible to uplink pressure against time data sets compensated for barometric pressure at the transmission site. The raw pressure trace data may not be displayed unless the physician is interested in monitoring the waveform. In the most likely cases the pressure data is reduced and normalized for the patient and displayed as a historical trend for the particular patient. There are numerous computed measurements that may be acquired from the pressure trace. These include the maximum dP/dt for the left ventricle; the systolic pressure; the beginning diastolic pressure (BDP); the mean diastolic pressure; the end diastolic pressure; the pulse pressure and the heart rate. This normal suite of calculations may be augmented or reduced for any particular patient. It is possible to allow for storage of up to two years of non-waveform data for each of 1000 patients, for example.

In a typical case, the physician will test the ITD 20 before implant by entering the pre-implant test state 1510. If the ITD 20 is appropriate and acceptable the ITD device will then be implanted. By invoking a state transition from the main menu the physician may set up the necessary data fields for a specific patient in state 1512.

The implanted device will typically be recalibrated periodically and the recalibration schedule may be kept on the PDCS 70. The physician may calibrate the ITD 20 and/or the HDCS 60 as such. For example the physician may recalibrate the ITD 20 from the calibrate state 1514. In one embodiment, a means is provided that allows the ITD 20 to be recalibrated as a result of independently obtained in vivo measurements of LV pressure. Recalibration will allow the telemetered measurements to return to within a accuracy of +/−5 mmHg for a period of 6 months following the recalibration procedure. This assumes that the means of independent measurements were obtained without error.

In general, the system 10 described herein provides a system for assessing the clinical status of CHF patient. The system includes a wireless implantable monitor for measuring pressure in the left ventricle (LV), electrical activity of the heart (electrogram—EGM), and temperature. The system also includes a device to receive the transmitted signal and record the transmitted signal or information derived therefrom. The system also includes a means for transferring data from the patient's location to the physicians location, as well as software to condense and display clinically relevant information to the physician.

Specifically, the system permits long-term assessment of pressure changes in any of the four chambers of the heart. The system is also designed for deriving information from the pressure data through mathematical manipulation of the pressure data. For example, it would be possible to derive systolic and diastolic pressures as well as heart rate. Further, it is possible to derive the positive and negative differentials of the pressures. The maximum positive and negative differentials are often used as measures of functioning of the left ventricle in animal studies. The measures are usually referred to in the literature as max LV+dP/dt, and max LV−dP/dt. It is not common to use these measures in the clinic since direct measurements of LV pressure are not commonly available.

Those skilled in the art will recognize that the present invention may be manifested in a variety of forms other than the specific embodiments described and contemplated herein. Accordingly, departures in form and detail may be made without departing from the scope and spirit of the present invention as described in the appended claims.

What is claimed is:

1. A method of implanting a pressure measurement device in a heart of a patient, comprising the steps of:
providing a pressure transducer assembly comprising a pressure transducer and a pressure transmission catheter, the catheter having a proximal portion and a distal portion, the proximal portion connected to the pressure transducer, the distal portion having an opening with a barrier; and
positioning the catheter across a heart wall, with the opening disposed in a chamber of the heart.

2. A method of implanting a pressure measurement device in a heart of a patient as in claim 1, further comprising the steps of:
providing an implantable telemetry unit;
connecting the telemetry unit to the pressure transducer assembly; and
implanting the telemetry unit in the patient.

3. A method of implanting a pressure measurement device in a heart of a patient as in claim 1, wherein the heart wall includes myocardium, and wherein the positioning step comprises positioning the catheter across the entire myocardium.

4. A method of implanting a pressure measurement device in a heart of a patient as in claim 1, wherein the chamber comprises a left ventricle, and wherein the positioning step comprises positioning the catheter across the heart wall with the opening disposed in the left ventricle.

5. A method of implanting a pressure measurement device in a heart of a patient as in claim 1, wherein the chamber comprises a right ventricle, and wherein the positioning step comprises positioning the catheter across the heart wall with the opening disposed in the right ventricle.

6. A method of implanting a pressure measurement device in a heart of a patient as in claim 1, wherein the heart wall comprises a ventricular septum, and wherein the positioning step comprises positioning the catheter across the ventricular septum.

7. A method of implanting a pressure measurement device in a heart of a patient as in claim 1, wherein the pressure transducer assembly includes a housing containing the pressure transducer, further comprising the step of securing the housing to the heart wall.

8. A method of implanting a pressure measurement device in a heart of a patient as in claim 7, wherein the securing step comprises securing the housing outside the heart.

9. A method of implanting a pressure measurement device in a heart of a patient as in claim 7, wherein the securing step comprises securing the housing inside the heart.

10. A method of implanting a pressure measurement device in a heart of a patient as in claim 1, wherein the positioning step comprises a surgical approach.

11. A method of implanting a pressure measurement device in a heart of a patient as in claim 1, wherein the positioning step comprises a translumenal approach.

12. A method of implanting a pressure measurement device in a heart of a patient as in claim 1, wherein the proximal portion is relatively crush-resistant and the distal portion is relatively flexible, and wherein the positioning step comprises positioning the relatively crush-resistant proximal portion in the myocardium and the relatively flexible distal portion in the chamber.

13. A method of implanting a device, comprising the steps of:
providing an implantable device comprising a telemetry unit connected to a pressure transducer assembly connected to a catheter; and
implanting the device such that the catheter extends across a heart wall, with a distal end of the catheter disposed in a chamber of the heart and the pressure transducer assembly connected to the heart wall outside the chamber.

14. A method of implanting a device as in claim 13, wherein the heart wall includes myocardium, and wherein the positioning step comprises positioning the catheter across the entire myocardium.

15. A method of implanting a device as in claim 13, wherein the chamber comprises a left ventricle, and wherein the positioning step comprises positioning the catheter across the heart wall with the opening disposed in the left ventricle.

16. A method of implanting a device as in claim 13, wherein the chamber comprises a right ventricle, and wherein the positioning step comprises positioning the catheter across the heart wall with the opening disposed in the right ventricle.

17. A method of implanting a device as in claim 13, wherein the heart wall comprises a ventricular septum, and wherein the positioning step comprises positioning the catheter across the ventricular septum.

18. An implantable pressure transducer assembly, comprising:
a pressure transducer; and
a liquid filled catheter, the catheter having a proximal portion and a distal portion, the proximal portion connected to the pressure transducer, the distal portion having an opening with a barrier, wherein the proximal portion is relatively crush-resistant and the distal portion is relatively flexible.

19. An implantable pressure transducer assembly as in claim 18, wherein the relatively crush-resistant proximal portion has sufficient crush resistance to prevent collapse caused by myocardial contraction.

20. An implantable pressure transducer assembly as in claim 19, wherein the catheter includes a metallic tube extending through the proximal portion and a polymeric tube extending through the distal portion.

21. An implantable pressure transducer assembly as in claim 20, wherein the polymeric tube extends through the proximal portion and the distal portion.

22. An imp lantable pressure transducer assembly as in claim 21, wherein the metallic tube is disposed in the polymeric tube.

23. An implantable pressure transducer assembly as in claim 19, wherein the opening is disposed at a distal end of the catheter.

24. An implantable pressure transducer assembly as in claim 19, wherein the opening is disposed proximal of a distal end of the catheter.

25. An implantable pressure transducer assembly as in claim 19, wherein the barrier comprises a gel.

26. An implantable pressure transducer assembly as in claim 19, wherein the barrier comprises a membrane.

27. A blood pressure measuring system for measuring blood pressure in a heart of a patient, comprising:
an implantable pressure transducer assembly;
an implantable telemetry unit connected to the pressure transducer assembly via an electrical lead;
a local data collection system in communication with the implantable telemetry unit via a wireless link; and
a remote data collection system in communication with the local data collection system via a telecommunication system.

28. A blood pressure measuring system as in claim 27, wherein the telecommunication system comprises the Internet.

29. A blood pressure measuring system as in claim 27, wherein the wireless link comprises radio transmission.

30. A blood pressure measuring system as in claim 27, wherein the pressure transducer assembly includes a pressure transducer and a liquid-filled catheter connected to the pressure transducer.

31. A blood pressure measuring system as in claim 30, wherein the pressure transducer assembly further includes a housing containing the pressure transducer, the housing having means for connection to the heart.

32. A blood pressure measuring system as in claim 31, wherein the liquid-filled catheter includes a proximal portion and a distal portion, the proximal portion connected to the pressure transducer, the distal portion having an opening with a barrier.

33. A blood pressure measuring system as in claim 32, wherein the proximal portion of the catheter is relatively crush-resistant and the distal portion of the catheter is relatively flexible.

34. A method of diagnosing or monitoring congestive heart failure (CHF) or an underlying cause thereof, comprising the steps of:
providing an implantable device comprising a telemetry unit connected to a pressure transducer connected to a catheter;
implanting the device such that the catheter extends across a heart wall, with a distal end of the catheter disposed in a chamber of the heart and the pressure transducer connected to the heart wall outside the chamber; and
monitoring blood pressure in the heart chamber using the implantable device.

35. A method of diagnosing or monitoring congestive heart failure in a patient comprising:
placing a fluid filled catheter across the myocardium of the patient;
coupling said fluid to an implanted pressure transducer mounted on the myocardium:
telemetering data from the pressure transducer to a remote location outside the body of the patient;
maintaining a data base of telemetered data over time to develop data trends for the patient;
displaying the data to a physician for diagnostic purposes.

36. The method of claim 35, wherein said telemtering step includes:
providing a base data collection station proximate the patient to communicate locally with said implanted pressure transducer;
providing a remote data collection station to communicate with said base station to collect store reduce and display patient data to a physician.

37. The method of claim 35, wherein said telemetry step includes:
providing an implantable telemetry unit coupled to a remote sensor module through a lead such that the sensor module may be affixed to the heart wall while the telemetry unit may be implanted abdominally or pectorally.

38. The method of claim 37, wherein said telemetry step includes:
providing a home data collection station that is communicates with said telemetry unit over a short range.

39. The method of claim 38, wherein said telemetry step includes:
providing a physician data collection station remote from said home station coupled to said home data collection station by a land based communication link.

40. The method of claim 35, wherein said displaying step includes:
a physician operated menu driven data display system with a first set of standard templates for displaying data and a second set of custom templates for displaying data.

41. The method of claim 38, wherein said telemetered data is collected in approximately real time and compensated for barometric variation during collection by said home data collection station creating barometric compensated pressure data.

42. The method of claim 35, wherein said maintaining and displaying steps includes calculating from said barometric compensated pressure data at least one value selected from the group consisting of:
LVSP/LVDP; LVBDP; LVEDP; HR; LVmax+dP/dT; LVmax−dP/dt; Pulse LVP; dP/dT.

43. The method of claim 42, wherein said calculating step includes:
maintaining a historical data set tracking computed variable for a patient over a time period of at least one month.
